(19) United States
(12) Patent Application Publication
(10) Pub. No.: US 2007/0104716 A1
(43) Pub. Date: May 10, 2007

(54) Methods for controlling pathological angiogenesis by inhibition of a6b4 integrin

(75) Inventor: Filippo Giancotti, New York, NY (US)

Correspondence Address: Marina Larson & Associates LLC;re: MSK, P. O. BOX 4928, DILLON, CO, 80435-4928, US

(73) Assignee: SLOAN-KETTERING INSTITUTE FOR CANCER RESEARCH, New York, NY (US)

(21) Appl. No.: 10/595,845

(22) PCT Filed: Nov. 22, 2004
(86) PCT No.: PCT/US04/39189

§ 371 Date: May 16, 2006

Related U.S. Application Data
(60) Provisional application No. 60/481,696, filed on Nov. 22, 2003.

(51) Int. Cl. A61K 48/00; A61K 39/395

(52) U.S. Cl. 424/155.1; 514/44

(57) ABSTRACT

It has now been determined that α6β4 integrin is a pro-angiogenic receptor, and thus that it provides a novel and heretofore unrecognized target for anti-angiogenic therapy. Thus, angiogenesis, particularly pathological angiogenesis, can be inhibited, and conditions with which pathological angiogenesis is associated treated using inhibitors of α6β4 integrin. A tissue in which angiogenesis is to be inhibited is exposed to a therapeutic agent effective to reduce the amount of active α6β4 integrin in the tissue. The tissue may be within a patient, and in particular a human patient to be treated for a disease condition with which pathological angiogenesis is associated. The therapeutic agent may be an antibody or a small molecule, for example a laminin-5 analog, which binds to α6β4 integrin and inhibits its normal function. The therapeutic agent may also be a chemical species that interferes with the production of α6β4 integrin, including for example an antisense or RNAi species.

This application claims the benefit of U.S. Provisional Application No. 60/481,696, filed Nov. 22, 2003, which is incorporated herein by reference.

### BACKGROUND OF INVENTION

This application relates to a method of inhibiting pathological angiogenesis, for example angiogenesis associated with cancer, diabetes, macular degeneration, and rheumatoid arthritis, through the inhibition of α6β4 integrin. In this application, the nomenclature α6β4 refers to the alpha-6-beta-4 integrin. Similar nomenclature with arabic or roman numerals is used for other integrins.

The integrin receptors constitute a family of cell surface proteins with shared structural characteristics of noncovalent heterodimeric glycoprotein complexes formed of alpha and beta subunits. There are eight known beta subunits and fourteen known alpha subunits, which associate in various combinations to form at least twenty-four receptors with different ligand specificities (Hynes, 2003). Integrins bind to extracellular matrix proteins, such as fibronectin, vitronectin, collagens and laminins, and to counter-receptors on other cells and therefore mediate cell-extracellular matrix and cell-cell interactions, referred generally to as cell adhesion events. Most integrins connect the extracellular matrix to the intracellular actin cytoskeleton and cooperate with Receptor Protein Tyrosine Kinases (RPTKs) to regulate cell fate (Giancotti and Ruoslahti, 1999; Miranti and Brugge, 2002). Depending on the integrins they express and the matrix they attach to, normal cells proliferate or undergo growth arrest, migrate or remain stationary, and live or undergo apoptotic death. These effects imply that the integrins impart a stringent control to the action of RPTKs, determining the nature and direction of the cell's response to growth factors and cytokines (Giancotti and Tarone, 2003). By regulating cell adhesion and signaling events, integrins play key roles in many biological processes, including development, tissue repair, hemostasis, inflammation, angiogenesis, and cancer. Various integrin antagonists have been discovered and developed for therapeutic purposes. Three inhibitors of the platelet integrin α11bβ3 have already received FDA approval and are currently used in the clinic to inhibit thrombosis. Inhibitors of αvβ3 and αvβ5 are being developed to treat tumor invasion, angiogenesis, and osteoporosis, and antagonists of β2 and α4 integrins to treat inflammatory and autoimmune diseases (Shimaoka and Springer, Nat Rev Drug Discovery, 2002).

The possibility of ameliorating, or even suppressing, the progression of cancer, diabetes, macular degeneration, and rheumatoid arthritis with anti-angiogenic drugs has attracted vivid interest (Kerbel R. Folkman J. Clinical translation of angiogenesis inhibitors. Nat Rev Cancer. 2002 October;2(10):727-39). Pathological angiogenesis is triggered by an enhanced production of pro-angiogenic factors, generally VEGF and bFGF, and/or a decreased generation of angiogenesis inhibitors. As a result, host vessels in the vicinity of the tumor or other source of angiogenic stimulus are destabilized and specific endothelial cells acquire an invasive phenotype. Upon detaching from adjacent cells and penetrating the underlying basement membrane, these cells proliferate and migrate as cords in the interstitial matrix. During the last phase of the process, the endothelial cells acquire a quiescent, differentiated phenotype: they deposit a basement membrane and acquire polarity, coincident with the formation of a lumen. Pericytes and smooth muscle cells are finally recruited to ensheat the newly formed vessels. These steps are repeated in an iterative manner, as mature vessels become locally destabilized and groups of endothelial cells re-acquire an invasive phenotype to generate a new vascular branch (reviewed in (Risau, 1997).

Known angiogenic factors, such as bFGF and VEGF, enhance the expression and activity of endothelial integrins (Byzova et al., 2000; Klein et al., 1993), whereas negative regulators of angiogenesis, such as class 3 semaphorins, promote vascular remodeling by inhibiting integrin function (Serini et al., 2003). it has been reported that the α5β1 integrin, a fibronectin receptor, and the αV integrins αvβ3 and AVOS, which bind to several RGD-containing matrix proteins, promote angiogenesis (reviewed in Hynes et al. (2002). This property has been considered as a basis for using inhibitors of such integrins as inhibitors of angiogenesis (See U.S. Pat. Nos. 5,981,478; 5,766,591; 6,358,970; and 6,645,991). However, while genetic experiments in mice have confirmed the role of α5β1 integrin in angiogenesis, they have not confirmed a role for the αV integrins, thus calling into question the efficacy of anti-angiogeneic therapy based on the latter group. Anti-angiogenic therapy based on inhibition of α5β1 integrin is problematic because of toxicity arising as a result of the critical involvement of this integrin in the adhesion of several cell types.

The α6β4 integrin is a laminin-5 receptor expressed by epithelial cells, Schwann cells, and endothelial cells and has several distinguishing features. The cytoplasmic domain of β4 is unusually long (ca. 1000 amino acids) and displays no homology to the short cytoplasmic tails of other β subunits. Upon α6β4 binding to matrix, the unique cytoplasmic domain of β4 is phosphorylated on multiple tyrosines by a Src Family kinase (SFK) and interacts directly with the signaling adaptor protein Shc, causing activation of the Ras to ERK cascade (Dans et al., 2001; Gagnoux-Palacios et al., 2003; Mainiero et al., 1995). In addition, the β4 tail mediates activation of PI-3K and Rac (Shaw, 2001; Shaw et al., 1997). Upon dephosphorylation, the cytoplasmic domain of β4 associates with the keratin cytoskeleton, causing assembly of hemidesmosomes and, hence, strengthening adhesion to laminin-5-containing basement membranes (Dans et al., 2001; Murgia et al., 1998; Spinardi et al., 1993). In contrast, the other integrins activate FAK/SFK signaling at focal adhesions (Geiger et al., 2001; Schlaepfer and Hunter, 1998) and, although some of them also recruit Shc, they do so by a distinct, indirect mechanism (Wary et al., 1998).

The α6β4 integrin has been studied predominantly in the context of epithelial and tumor biology studies. In stratified and transitional epithelia, α6β4 mediates, upon cessation of signaling, assembly of hemidesmosomes (Dans et al., 2001; Murgia et al., 1998; Spinardi et al., 1993). Activation of the EGF-R and Ron RTKs enhances phosphorylation of β4, causing disruption of hemidesmosomes and increased epithelial cell migration (Dans et al., 2001; Santoro et al., 2003; Trusolino et al., 2001), suggesting that these RTKs decrease the ability of α6β4 to mediate stable adhesion but increase its signaling function.

Many invasive carcinomas display elevated levels of (α6β4 (reviewed in Mercurio and Rabinovitz, 2001). Introduction of α6β4 in breast and colon carcinoma cells that have lost its expression activates PI-3K to Rac signaling and increases invasive ability in vitro (Shaw et al., 1997). In addition, the β4 tail functions as an essential adapter and amplifier of pro-invasive signals elicited by activated Met in cells undergoing Met-induced oncogenesis Trusolino et al., 2001). Finally, introduction of a dominant negative form of β4 impairs the survival of breast carcinoma cells, and this effect has been linked to the ability of mutant β4 to interfere with the assembly of hemidesmosomes and the establishment of a partially polarized phenotype (Weaver et al., 2002). Collectively, these results suggest that α6β4 promotes carcinoma invasion and growth (Gambaletta et al., 2000; Trusolino et al., 2001).

The observation that β4-null embryos do not display defective vasculogenesis or developmental angiogenesis (Dowling et al., 1996; van der Neut et al., 1996) suggested no role for α6β4 during angiogenesis.

### SUMMARY OF INVENTION

It has now been determined that the α6β4 integrin is a pro-angiogenic receptor, and thus that it provides a. novel and heretofore unrecognized target for anti-angiogenic therapy. Thus, the present invention provides methods for the inhibition of angiogenesis, particularly pathological angiogenesis, and for the treatment of conditions with which pathological angiogenesis is associated, using inhibitors of the α6β4 integrin. In accordance with the method of the invention, a tissue in which angiogenesis is to be inhibited is exposed to a therapeutic agent effective to reduce the amount of active α6β4 integrin in the tissue. In one embodiment of the invention, the tissue is within a patient, and in particular a human patient, to be treated for a disease condition with which pathological angiogenesis is associated. The therapeutic agent may be an antibody, a molecular recognition scaffold, a cyclic peptide or a peptidomimetic, for example a laminin-5 analog, or a small molecule, which bind to the α6β4 integrin and inhibits its normal adhesive and signaling functions. The therapeutic agent may bind to the α6β4 integrin and inhibits only its normal signaling function, leaving its normal adhesive function unaffected. The therapeutic agent may also be a chemical species that interferes with the production of α6β4 integrin, including for example an antisense or RNAi species. The therapeutic agent is administered to the tissue or patient in a therapeutically effective amount and may be used in combination with other anti-angiogenesis therapies, or other established anti-cancer therapies such as chemotherapy, radiation therapy, or surgery.

### BRIEF DESCRIPTION OF THE DRAWINGS

FIG. 1 shows a replacement vector, wild-type locus, and mutant locus. Solid boxes: exons. TM: exon encoding the transmembrane segment. Open boxes: cDNA sequences, solid asterisk: stop codon, polyA: SV40 polyadenylation signal, neo: Neomycin resistance cassette, TK: thymidine kinase, E: EcoRi, N: Ncol, p5′ and p3′: probes for Southern blotting. Wild-type protein (β4 WT) and truncated mutant (β4 1355T) are shown below. White boxes: fibronectin type-III repeats, open asterisks: tyrosine phosphorylation sites.

FIG. 1 B shows results when wild-type and mutant keratinocytes were subjected to FACS analysis with mAb 346-11A, which binds to the extracellular domain of mouse β4.

FIG. 1 C shows a graph of adhesion as a function of Ln-5 concentration. Wild-type (WT) and mutant (1355T) keratinocytes were plated for 1 hour on microtiter plates coated with the indicated amounts of laminin-5 at 4° C. Cell adhesion to fibronectin at 4° C. was negligible.

FIG. 2A shows results when bFGF containing plugs from FITC-Lectin injected wild-type and mutant mice were lysed and subjected to fluorimetry. The graph shows the mean (±SD) from three experiments (*, P<0.003).

FIG. 2 B shows quantification of vascular glomeruli abutting the limiting membrane in wild-type (WT) and mutant (1355T) retinas (n=5 mice per genotype)(*, P<0.004).

FIG. 3 shows results when sections of bFGF containing plugs from wild-type and mutant mice injected with BrdU were subjected to double staining with anti-PECAM-1 (red) and anti-BrdU (green) (left). The graph shows the mean (±SD) number of BrdU+ cells per 100 vessel cross-sections examined (right).

FIG. 4A shows results when HUVECs (Ctrl) and HUVECs transfected with α6 in combination with either wild-type β4 or β4-1355T were plated on laminin-5 and induced to migrate across an artificial wound in response to bFGF. When indicated, migrating cells were treated with the NF-kB inhibitor BAY 11-7082 (12.5PM) or the MAPK inhibitor PD98059 (50 μM). The graph shows the mean percentage of wound closure at 8 hours (±SD) from three experiments (*, P<0.001 versus β4-1355T, P<0.002 versus Ctrl or BAY 11-7082 inhibitor and P<0.003 versus PD98059 inhibitor).

FIG. 4B shows results when HUVECs and the indicated derivatives were grown on Cytodex-3 beads and placed in collagen gels containing bFCF for 72 hours. The graph indicates the average number (±SD) of cord-like structures emanating from each bead (*, P<0.002. versus Ctrl and P<0.001 versus β4-1355).

FIG. 5A shows result when B16F0 melanoma, LLCL Lewis Lung Carcinoma, B6RV2 lymphoma, and 60.5 fibrosarcoma cells were injected s.c. in wild-type or □□□mutant mice. The YD-Neu mammary carcinoma cells were injected orthotopically in MMTV-Neu mice expressing wild-type or mutant □□ to avoid an Immune response to rat Neu. The graphs show mean tumor volumes (±SD) after 10 days (60.5), 12 days (B16F0 and LLCI), 13 days (B6RV2) or 20 days (YD-Neu) (*, P<0.004.in B16F0, P<0.09 In LLCI, P<0.01 in B6RV2).

FIG. 5B shows results when sections of the indicated tumor xenografts from wild-type and mutant mice were stained with anti-PECAM-1 antibodies. The graphs show the average microvessel densities (±SD) in each tumor. Ten random high power fields per tumor section were evaluated. Bar: 200 pm (*, P<0.01 in B16F0, P<0.004 in LLCI, P<0.02 in B6RV2 and P<0.005 in 60.5).

FIG. 5C shows results when confocal images of B16F0 melanoma tumors excised from FITC-Lectin injected wild-type and mutant mice. The graph shows the average number of branches (±SD) per high power field for each tumor (*, P<0.02). Bar: 100 μm.

FIG. 6 shows a hypothetical model of α6β4 function in angiogenesis

### DETAILED DESCRIPTION

As used in this application, the term “pathological angiogenesis” refers to angiogenesis which is considered medically abnormal and suitable for therapeutic treatment. Pathological angiogenesis occurs in a variety of disease conditions, including without limitation cancer, diabetes, rheumatoid arthritis, and eye diseases such as macular degeneration(Folkman, Nat. Med. 1995: Kerbel and Folkman, Nat. Rev. Cancer 2002). The term cancer refers to pathological growths, both benign and malignant, of a variety of tissue origins and histological apperances, including without limitation colorectal cancer, breast cancer, prostate cancer, pancreatic cancer, lung cancer, mesothelioma, melanoma, osteosarcoma, hepatocellular carcinoma, hemangioblastoma, renal cell carcinoma, Kaposi's sarcoma, gastrointestinal stromal tumor (GIST), Glioblastoma Multiforme (GBM) and other brain tumors, myeloma and other hematopoietic malignancies such as chronic and acute leukemias and lymphomas, myelodisplastic syndromes such as myelofibrosis and essential thrombocytopenia, squamous carcinoma of the skin, head and neck cancer, thyroid cancer, cervical cancer, leyomyomata of the uterus, and pulmonary hemangloma. The term diabetes refers to both type I and II and applies especially to diabetic retinopathy. Age-related macular degeneration refers to both wet and dry. In addition to Rheumatoid Arthritis, inflammatory joint and skin diseases such as osteoarthritis and psoriasis are also included. Angiogenesis-dependent diseases also include obesity, the hereditary Von-Hippel-Lindau cancer predisposition syndrome (VHL), and Hereditary Hemorragic Teleangiectaesia (HHT).

As used in this application, the term “inhibition” refers to a reduction of the event or activity inhibited to an extent sufficient to produce an observable result. Complete elimination of the event or activity is not required.

As used in this application, the term “amount of active α6β4 integrin” refers to the observable angiogenesis-promoting activity resulting from α6β4 integrin present in a tissue. Reductions in the amount of the active α6β4 integrin can result from a reduction in the amount of α6β4 integrin, i.e, effectively a reduction in concentration; a reduction in the capacity of individual molecules of α6β4 integrin to promote angiogenesis, i.e. effectively a change in the quality of the integrin, or combinations thereof. The first type of reduction will most commonly be achieved by limiting the production of α6β4 integrin, for example using a antisense oligonucleotide or RNAi techniques, although it could also be achieved by accelerating the decomposition of α6β4 integrin. The second type of reduction is most readily achieved through physical binding of the integrin with a molecule that competes with its normal ligand for binding to the integrin, or inhibits the conformational changes, which underlie signal transduction accross the membrane—both inside-to-outside signaling (activation) and outside-to-inside signaling, or that inhibit association of the integrin with RPTKs, or that competes with those intracellular molecules that bind to the cytoplasmic tail of the integrin and promote signaling events.

As used in this application, the terms “treatment” or “treating” refer to the application of a therapeutic agent to achieve a reduction in the amount of active α6β4 integrin so as to produce a benefit to a patient being treated. Such a benefit need not be a complete or permanent cure, but may be only a lessening of the rate at which angiogenesis is occurring, thereby delaying progression of a disease condition.

As used in this application, the term “administration” refers to any means by which a therapeutic agent can be delivered to a tissue, including without limitation oral, nasal and transdermal administration and injection, for example subcutaneous, subdermal, intramuscular, intravenous, intrathecal or peritoneal injection. For treatment of eye disease with associated angiogenesis, methods include without limitation direct injection into the eye, trans-scleral administration with a patch, or implanted slow-release pellets.

The effective amount of a therapeutic agent to be administered varies depending on the nature of the therapeutic agent, and will frequently reflect a balancing of therapeutic benefits and side effects. However, the determination of specific amounts or a given therapeutic is routine and within the skill in the art. The therapeutic agent may be used as single therapy or in combination with other direct inhibitors of angiogenesis, including without limitation Angiostatin, Bevacizumab (Avastin), PTk787, ZD6474, SU6668, SUl 1248, Arresten, Canstatin, Combrestatin, Endostatin, NM-3, Thrombospondin, Tumstatin, 2-methoxyestradiol, Vitaxin, Thalidomide and TNP-470. The therapeutic agent may also be used in combination with “indirect” inhibitors of angiogenesis, which inhibit production of pro-angiogenic factors by tumor cells, including without limitation ZD1839 (IRESSA), OS1774 (Tarceva), IMC225 (Erbitux), Herceptin, IFN-a, and metronomic chemotherapy (Kerbel and Folkman, Nat. Rev. Cancer, 2002).

Therapeutic agents useful in the present invention may be antibodies, engineered binding proteins, endogenous binding proteins, RNA- or DNA-aptamers or small molecules that bind to α6β4 integrin to produce a reduction in activity. The term antibodies includes full length antibodies (including naturally occurring antibodies and engineered antibodies), and antigen binding fragments of naturally occurring or engineered antibodies. The antibodies may be chimeric, CDR-7grafted, humanized, deimmunized, as well as other antibody molecules which have been engineered to reduce immunogenicity, e.g., those having CDRs derived from a non-human source, e.g., derived from an nonhuman animal such as a mouse, and or derived from random or partially random generation of sequences, e.g., a by use of a phage display method. Such non-human can be inserted into human, humanized, or other frameworks which are less antigenic when administered to a human. The antigen binding fragments of naturally occurring or engineered antibodies may include single chain antibodies, intrabodies, and bi-valent antibodies. The term molecular recognition scaffolds includes protein fragments modeled after transferrin (Trans-bodies—BioRexis)), fibronectin (Tetranectins—Borean Pharma; AdNectins—Compound Therapeutics), S. Aureus Protein A (Affibodies—Affibody), or other proteins or protein modules engineered to bind to α6β4. Such antibodies may be monoclonal, polyclonal, or modified constructs, for example single chain Fv constructs, Fab fragments, single domain antibodies, or bispecific antibodies, targeting α6β4 integrin. Binding sites may be on the alpha chain, the beta chain or both chains of the α6β4 integrin.

Other examples of therapeutic agents include small molecules which block β4 signalling by binding to β4, and have specific functions such as inhibiting nuclear translocation of NF-kB. Where an antibody therapeutic agent is used, it may be administered in the form of the antibody, or formed in situ by expression of a nucleic acid sequence encoding an α6β4 integrin-specific antibody.

Non-antibody binding proteins could also be employed. For example, human integrin-beta-4 binding protein is known and has the sequence given by Seq. ID No. 1 which is known fron GenBank accession no. NM_{—}002212.

The therapeutic agent may also be a nucleic acid that results in a reduction in the amount of active α6β4 integrin, for example an antisense oligonucleotide or an RNA molecule that works by an RNAi mechanism. The nucleic acid may target, via a sequence specific mechanism, the alpha chain or the beta chain. The coding sequence of the beta 4 chain of human integrin is known from NM_{—}000213 to be as shown in Seq. ID No. 2. The coding sequence of the alpha 6 chain of human integrin is known from NM_{—}000210 to be as shown in Seq ID No. 3.

Antisense and RNAi sequence are derivable from these sequences. Antisense oligonucleotides are commonly from 12 to 50 bases in length, more preferably 15-30 bases length. Effective regions for targeting of antisense sequences may be found throughout the target nucleic acid. A preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon⇄ or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding integrin beta 4, regardless of the sequence(s) of such codons.

It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5-5′ triphosphate linkage. The 5′ cap region of an mRNA is considered to include the 5′ cap structure itself as well as the first 50 nucleotides adjacent to the cap. The 5′ cap region may also be a preferred target region.

RNAi molecules are similarly selected based on the sequence and defined parameters known for the selection of appropriate sequences. RNAi molecules may be single or double stranded, and generally have a length of 19 to 23 bases, although longer and shorter species can be used. A specific RNAi species useful in the method of the invention is based on the mouse sequence of beta-4 cDNA (Genebank Acc. # L04678): nucleotides 113 to 131; counting from the A of the ATG translational start site, having the sequence GAGCTGTACCGAGTGCATC (Seq. ID. No. 4). This molecule, and the corresponding molecule based on the human sequence, and their use form a further aspect of this invention.

Inhibition of α6β4 integrin may also be achieved using an shRNA sequence GAGCUGCACGGAGUGUGUC (Seq. ID No. 5) described in Chung et al., “integrin (alpha 6 beta 4) regulation of eIF-4E activity and VEGF translation: a survival mechanism for carcinoma cells” J Cell Biol. 2002 Jul. 8;158(1):165-74. Epub 2002 Jul. 08.

Inhibition of α6β4 integrin may be achieved using a murine sequence AAGAGCTGTACCGAGTGCATC (Seq. ID. No. 6). Delivery of this sequence is suitably done using the procedure and vector set forth in Barton et al. “Retroviral delivery of small interfering RNA into primary cells.” Proc Natl Acad Sci USA. 2002 Nov. 12;99(23):14943-5. Epub 2002 Nov. 04.

The anti-alpha-6 Mab CoH3 blocks both human and mouse a6b1 and a6b4 And may be useful in this invention. Niessen et al., Exp Cell Res. 1994 Apr.;211 (2):360-7). The anti-beta-4 Mab ASC-3 blocks human beta-4 (Weaver et al. “beta4 integrin-dependent formation of polarized three-dimensional architecture confers resistance to apoptosis in normal and malignant mammary epithelium” Cancer Cell. 2002 September;2(3):205-16). Another known antibody that may be used in the present invention is anti-beta-4 Mab 346-11A blocks mouse beta-4 (Zent et al. “Involvement of laminin binding integrins and laminin-5 in branching morphogenesis of the ureteric bud during kidney development.” Dev Biol. 2001 Oct. 15;238(2):289-302).

Screening Assays

The invention provides methods (also referred to herein as “screening assays”) for identifying modulators (e.g., proteins, peptides, peptidomimetics, peptoids, small molecules, aptamers or other drugs) which bind to integrin α6β4 or the integrin β4 chain cytoplasmic tail, and have a stimulatory or inhibitory effect on, for example, integrin α6β4 adhesive or signaling function. Compounds thus identified can be used to modulate the activity of integrin α6β4 adhesion or signaling in a therapeutic protocol.

In one embodiment, the invention provides assays for screening candidate or test compounds that bind to or modulate an activity of integrin α6β4 e.g., compounds that modulate the adhesive function of integrin α6β4, or compounds that modulate the signaling function of the integrin β4 chain signaling function.

In one embodiment, an assay is a cell-based assay in which a cell which expresses integrin α6β4 is contacted with a test compound, and the ability of the test compound to modulate integrin α6β4-dependent adhesion or signaling is determined. Determining the ability of the test compound to modulate integrin α6β4 adhesion can be accomplished by monitoring the interaction of integrin α6β4 with its ligand Laminin-5. Determining the ability of the test compound to modulate integrin α6β4 signaling activity can be accomplished by monitoring, for example, activation PI-3K, ERK, JNK and NF-kB in cells adhering to laminin-5 (Mainiero et al. 1995; 1997; Dans et al., 2001; Nikolopoulos et al. 2004).

The ability of the test compound to modulate purified integrin ct6β4 binding to a compound, e.g., an integrin α6β4 ligand such as Laminin-5, or to modulate purified integrin β4 chain cytoplasmic tail signaling, can also be evaluated.

In yet another embodiment, a cell-free assay is provided in which integrin α6β4 or the β4 chain cytoplasmic tail is contacted with a test compound and the ability of the test compound to bind to the integrin α6β4 or the β4 cytoplasmic tail is evaluated.

Cell-free assays involve preparing a reaction mixture of the target integrin α6β4 or the β4 chain cytoplasmic tail protein and the test compound under conditions and for a time sufficient to allow the two components to interact and bind, thus forming a complex that can be removed and/or detected.

In one embodiment, determining the ability of the integrin α6β4 or the β4 chain cytoplasmic tail to bind to a target molecule can be accomplished using real-time Biomolecular Interaction Analysis (BIA) (see, e.g., SJolander, S. and Urbaniczky, C. (1991) Anal. Chem. 63:2338-2345 and Szabo et al. (1995) Curr. Opin. Struct. Biol. 5:699-705). “Surface plasmon resonance” or “BIA” detects biospecific interactions in real time, without labeling any of the interactants (e.g., BIAcore).

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., (1993) Trends Biochem Sci 18:284-7); chromatography (gel filtration chromatography, ion-exchange chromatography); electrophoresis (see, e.g., Ausubel, F. et al., eds. Current Protocols in Molecular Biology 1999, J. Wiley: New York.); and Immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., (1998) J Mol Recognit 1:141-8; Hage, D. S., and Tweed, S. A. (1997) J Chromatogr B Biomed Sci Appl. 699:499-525). Further, the interaction between two molecules can also be detected, e.g., using fluorescence energy transfer (FET) (see, for example, Lakowicz et al., U.S. Pat. No. 5,631,169; Stavrianopoulos, et al., U.S. Pat. No. 4,868,103), to detect binding without further purification of the complex from solution.

In a preferred embodiment, the assay includes contacting the integrin (α6β4 or the integrin β4 chain cytoplasmic tail with a library of small molecules, and separating them by high performance gel filtration [Affinity Selection: An Emerging Technology for Drug Discovery”, Satish Jindal and George R. Lenz, Drug Discovery and Design, Decision Resources, Waltham, Mass., No. 20, 1-13 (1998)].

The present invention was developed as part of an investigation into the physiological function of α6β4 signaling. Recent studies have indicated that α6β4 associates with multiple RPTKs, including the EGF-R, ErbB2/Neu, Met, and Ron (Gambaletta et al., 2000; Mariotti et al., 2001; Santoro et al., 2003; Trusolino et al., 2001). Activation of the EGF-R and Ron enhances phosphorylation of β4, causing disruption of hemidesmosomes and increased epithelial cell migration (Mariotti et al., 2001; Santoro et al., 2003; Trusolino et al., 2001). These results suggest that these RPTKs decrease the ability of α6β4 to mediate stable adhesion but increase its signaling function. Deregulation of α6β4/RPTK co-signaling may contribute to carcinoma invasion and growth (Gambaletta et al., 2000; Trusolino et al., 2001). The observation that β4-null embryos do not display defective vasculogenesis or developmental angiogenesis (Dowling et al., 1996; van der Neut et al., 1996) suggested no role for α6β4 during angiogenesis. Nevertheless, the Inventor made the hypothesis that α6β4 participates in pathological angiogenesis. Mice carrying a targeted deletion of the entire cytoplasmic domain of β4 lack hemidesmosomes and, like β4-null mice, die at birth due to extensive blistering of the skin and upper gastrointestinal tract (Murgia et al., 1998). To analyze the role of α6β4 signaling in the absence of the effect of loss of adhesion strengthening, we have generated mice carrying a deletion of the C-terminal, signaling segment of the β4 tail. Through an analysis of these mice and cells derived from them, we found evidence that α6β4 signaling controls epidermal growth and pathological angiogenesis through a previously unrecognized effect on nuclear translocation of key transcriptional regulators.

Targeted Deletion of the Integrin β4 Substrate Domain Impairs Signaling to ERK and AKT

Two developments made it possible to address the role of β4 signaling in postnatal life in the absence of potentially confounding effects of loss of adhesion. First, it became clear that the N-terminal part of the cytoplasmic domain of β4 to amino acid 1355 is sufficient for interaction with the plain HD-1/pectin and, hence, for association with the keratin cytoskeleton (Schaapveld et al., 1998). Second, mapping studies revealed that the five major tyrosine phosphorylation sites of β4, including those involved in the recruitment of Shc and PI-3K, are located in the C-terminal portion of the β4 tail, downstream of amino acid 1355 (Dans et al., 2001). We thus reasoned that a deletion of the C-terminal portion of the β4 cytoplasmic domain (henceforth defined “substrate domain”) would suppress α6β4 signaling without interfering with adhesion strengthening.

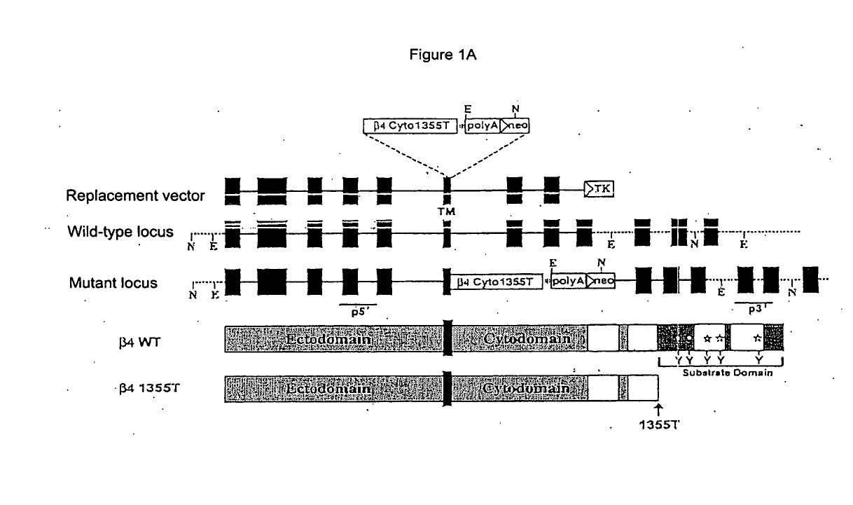

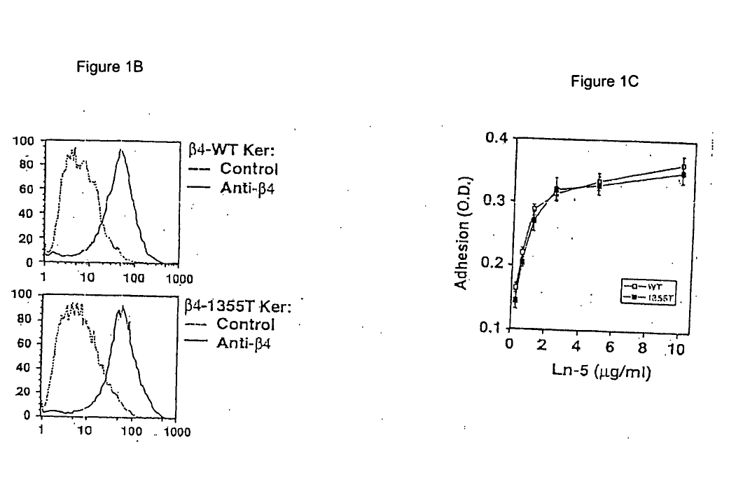

We used homologous recombination in ES cells to introduce such a mutation in mice. To construct the vector, we cloned the sequences encoding the cytoplasmic domain of β4 up to amino acid 1355, a stop codon, a SV40 polyadenylation signal, and a neomycin resistance gene, immediately downstream of the exon encoding the transmembrane segment of the protein (FIG. 1A). Southern blotting and PCR analysis indicated successful introduction of the mutation in mice. Analysis of the intercrosses between heterozygous mice carrying the targeted deletion revealed that the mutation was transmitted with the expected Mendel Ian frequency. Both homozygous and heterozygous β4 mutant mice were found to be viable, fertile, and to not manifest skin fragility. Histological analysis of the skin did not reveal any defect in epidermal adhesion to the basement membrane. Thus, deletion of the signaling domain of β4 has no obvious effect on embryonic and postnatal development.

Immunoprecipitation and FACS analysis on primary keratinocytes from wild-type and mutant mice indicated that the β4-1355T subunit associates with β4 and is expressed at the cell surface as well as wild-type β4 (FIG. 1 B). To test the adhesive ability of the mutant integrin, wild-type and mutant keratinocytes were plated on laminin-5 at 4° C. At this temperature, the function of α3β1—which also binds to laminin-5—is inactivated and adhesion proceeds only through ct⁶β⁴(Gagnoux-Palacios et al., 2003; Xia et al., 1996). The mutant keratinocytes attached to laminin-5 at 4° C. as efficiently as wild-type keratinocytes, suggesting that the mutant integrin retains intact ligand binding capacity (FIG. 1 C). In accordance with the absence of a skin fragility phenotype. transmission electron microscopy (EM) revealed that the skin of mutant mice contained well-structured hemidesmosomes. Thus, deletion of the C-terminal, signaling domain of β4 does not affect the ability of (α6β4 to establish a transmembrane connection between laminin-5 and the hemidesmosomal cytoskeleton and to mediate stable epidermal adhesion in vivo.

To examine the effect of deletion of the β4 substrate domain on signaling, primary keratinocytes isolated from wild-type and mutant mice were plated on laminin-5 or, as a control, on collagen I in the presence of serum and subjected to immunoblotting with anti-phospho-ERK and anti-phospho-AKT antibodies. Adhesion to laminin-5 induced significant phosphorylation of ERK in wild-type but not in mutant keratinocytes, whereas adhesion to collagen I caused similarly high activation of ERK in both types of cells. This result is consistent with the role of the β4 substrate domain recruitment of Shc and activation of Ras to ERK signaling (Dans et al., 2001; Mainiero et al., 1997). In addition, adhesion to laminin-5 led to significant phosphorylation of AKT in wild-type keratinocytes, but it induced a much more limited effect in mutant keratinocytes, in agreement with the hypothesis that the β4 substrate domain activates PI-3K to AKT signaling (Shaw et al., 1997). We concluded that targeted deletion of the C-terminal segment of the β4 tail impairs α6β⁴-dependent signaling through ERK and AKT, but it does not affect adhesion to laminin-5 and assembly of hemidesmosomes

α6β4 and its Ligand Laminin-5 Are Expressed in Tumor Vasculature

The mutant mice did not display any macroscopic defect suggestive of defective cardiovascular development, indicating that α6β4 signaling does not play an essential role during embryonic vasculogenesis and angiogenesis. This conclusion is consistent with the observation that α6β4 is expressed in blood vessels only after completion of developmental angiogenesis (Hiran et al., 2003). To examine the potential role of α6β4 in tumor angiogenesis we first studied the expression of α6β4 in paraffin-embedded sections of human papillary thyroid carcinoma, breast adenocarcinoma, prostate carcinoma, and glioblastoma multiforme. Significant levels of α6β4 were detected in medium and small-size vessels in all these tumors. Since tumor cells in breast and prostate cancer samples expressed high levels of α6β4, these samples were subjected to anti-PECAM-1 staining to unequivocally identify tumor vessels.

To further characterize the expression of α6β4 during tumor angiogenesis, we examined frozen sections of B16F0 melanoma xenografts. Double staining with antibodies to β4 and PECAM-1 showed that α6β4 is expressed in these tumors in medium and small size vessels, but not in microvessels. The anti-β4 antibodies also reacted with structures resembling peripheral nerves. Double staining with antibodies to β4 and to the neurofilament protein S-100 confirmed the identification of these structures as peripheral nerves. This observation is consistent with the known expression of (α6β4 in Schwann cells (Einheber et al., 1993) and the increasing evidence that tumors, including melanoma, are innervated (Seifert and Spitznas, 2002). Notably, the anti-β⁴ antibodies also stained vessel-like structures that reacted with anti-PECAM-1 very weakly. These structures reacted with antibodies to the lymphatic endothelial hyaluronan receptor (LYVE-1), suggesting that α6β4 is also expressed in tumor lymphatics.

To examine if the expression of β4 in endothelial cells correlated with the presence of vascular smooth muscle cells, we subjected the tumor sections to double staining with antibodies to β4 and to smooth muscle α-actin. Approximately half of the β4+ vessels were found to be ensheated by smooth muscle cells, whereas the remainder was not, suggesting that endothelial cells do not express β4 in response to a signal generated by mural cells. Significant amounts of laminin-5 were detected in the basement membrane of both β4+ medium and small size vessels and β4-microvessels, suggesting the existence of another laminin-5-binding integrin in these smaller vessels. In fact, the staining patterns generated by anti-laminin-5 and anti-PECAM-1 antibodies were virtually identical. Antibodies to α6 decorated all PECAM-1+ vessels, irrespective of β4 expression, indicating that the β4-microvessels express α6β1, It is possible that α6β1 or another laminin-binding integrin, such as alp1, mediates endothelial cell adhesion to laminin-5 in microvessels. These results indicate that the endothelial cells of tumor vessels deposit and organize a laminin-5-rich basement membrane and, as they mature, attach to it through α6β4.

The β4 Substrate Domain Promotes bFCF and VEGF-Mediated Angiogenesis

To examine if α6A4 signaling plays a role in bFGF-induced angiogenesis, Matrigel plugs containing bFGF were implanted in wild-type and mutant mice and recovered 7 days later. Macroscopic analysis revealed that the plugs from mutant mice were much paler than those from control mice. To visualize the development of vascular ramifications in the plugs, the mice were injected with an endothelial-specific FITC-labeled Lectin prior to euthanasia. Confocal analysis indicated that the vascular tree was in mutant plugs much less developed and complex than in wild-type plugs. The medium-size vessels penetrating into these plugs generated significantly fewer branches than expected, and these secondary branches only occasionally formed tertiary ramifications. Fluorimetry indicated that the mutant plugs had incorporated approximately five fold less FITC-Lectin than wild-type controls (FIG. 2A). In addition, immunoblotting showed that the mutant plugs contained a much smaller amount of VEGF-R and, by inference, of angiogenic endothelial cells than wild-type plugs. These observations indicate that loss of β4 signaling impairs bFGF-induced angiogenesis to a significant extent.

We examined if α6β4 signaling is required for angiogenesis in the retinal neo-vascularization model. In this model, angiogenesis is driven by hypoxia-induced production of VEGF (Shweiki et al., 1992). P7 mice were maintained in 75% oxygen for 5 days to induce central avascularization in the retina and then returned to normoxic conditions for 5 additional days. Histological analysis indicated that numerous vascular glomeruli penetrated the inner limiting membrane and abutted in the vitreous in wild-type mice, whereas the development of these abnormal vessels was significantly blunted in mutant mice. Quantification of the results confirmed that mutant mice have a significantly reduced angiogenic response to retinal hypoxia (FIG. 2B). Taken together, these results indicate that α6β4 signaling promotes both bFGF and VEGF-induced angiogenesis. α6β4 signaling is not required for endothelial proliferation or survival

To examine the cellular mechanism by which α6β4 signaling regulates angiogenesis, we conducted immunohistochemical studies on Matrigel plugs from wild-type and mutant mice. Anti-PECAM-1 staining of frozen sections showed that the angiogenic vessels of mutant mice penetrated significantly less into the bFGF-containing Matrigel plugs than those of wild-type mice. The wild-type plugs contained two-types of vessels: small-size vessels, which were detected predominantly at the periphery of the plug, and microvessels, which penetrated inside the plug. By contrast, the mutant plugs contained almost exclusively peripheral small-size vessels, and these were somewhat reduced in number as compared to those of wild-type plugs. While the endothelial cells of small-size vessels expressed α6β4, those of microvessels did not express the integrin. These observations suggest that deletion of the β4 substrate domain interferes with the sprouting of β4+small size vessels into β4-microvessels.

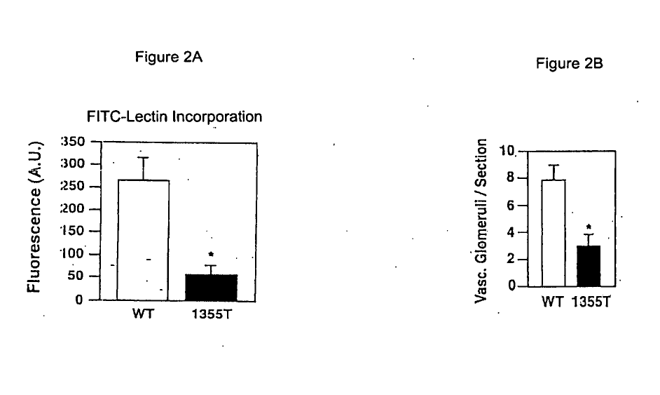

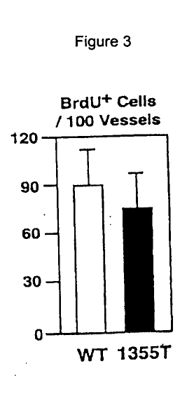

We next evaluated endothelial cell survival and proliferation in Matrigel plugs from wild-type and mutant mice. Anti-BrdU staining revealed that the number of endothelial cells in S-phase was significantly reduced in the plugs from mutant mice. However, the number of BrdU+ nuclei per PECAM-1+ vessel was similar in wild-type and mutant plugs, suggesting that the overall reduction of BrdU staining in the plugs of mutant mice was secondary to reduced sprouting, and it was not due to an intrinsic proliferative defect (FIG. 3). In addition, the small size vessels, which express β4, displayed very few BrdU nuclei as compared to the smaller PECAM-1+β4-capillaries, indicating that α6β4 is expressed in quiescent vessels. These observations suggest that signaling by the β4 substrate domain is not required for endothelial proliferation during angiogenesis. TUNEL staining did not reveal endothelial cell apoptosis in either wild-type or mutant plugs, suggesting that β4 signaling is not required for endothelial cell survival during angiogenesis. Together with the pattern of expression of α6β4 during angiogenesis, these results suggest that α6β4 signaling promotes the onset of the invasive phase of angiogenesis. These observations are consistent with the hypothesis that α6β4 functions at a step of angiogenesis that precedes overt endothelial cell proliferation and migration in the interstitial matrix.

The α6β4 Substrate Domain Promotes Endothelial Migration and Invasion

To examine the effect of α6β4 signaling on endothelial migration and invasion, we isolated endothelial cells from the lungs of wild-type and mutant mice. However, both types of cells lost expression of α6β4 upon plating in culture. We note that endothelial cells migrating out of human saphenous vein explants also lose expression of α6β4 (Hiran et al., 2003). We thus used transient transfection to introduce wild-type or mutant α6β4 in Human Umbilical Vein Endothelial Cells (HUVECs), as reported previously (Dans et al., 2001). HUVECs, which express almost undetectable levels of endogenous α6β4, were electroporated with plasmids encoding α6 and either wild-type β4 or mutant β4-1355T and panned on anti-β⁴ coated plates to isolate cells expressing comparable levels of recombinant wild-type or mutant α6β4, respectively.

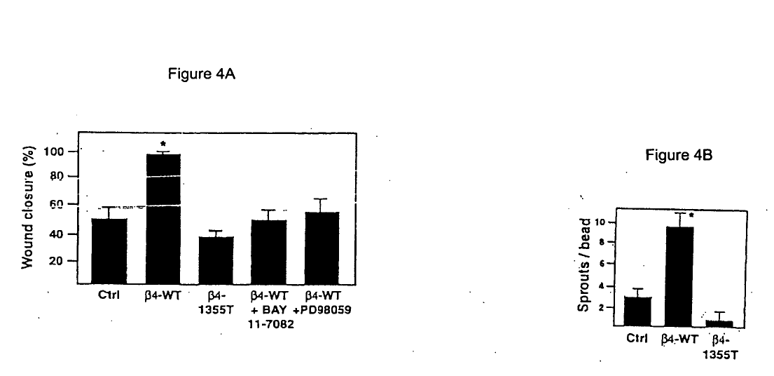

To examine the effect of α6β4 signaling on endothelial cell migration, parental HUVECS and their derivatives expressing either wild-type or mutant α6β4 were plated on laminin-5 at confluency and subjected to in vitro wound assay. Expression of wild-type α6β4 increased endothelial cell migration in response to bFGF. By contrast, the mutant integrin did not cause this effect (FIG. 4A), indicating that the β4 substrate domain promotes endothelial cell migration.

To evaluate the effect of α6β4 signaling on endothelial cell invasion, HUVECs expressing wild-type or mutant β4 were grown on Cytodex-3 beads and then incubated in collagen gels containing bFGF. Over a 3-day period, the endothelial cells expressing wild-type β4 migrated radially out of the beads and assembled into cords invading the collagen gel. By contrast, both control cells and cells expressing mutant β4 invaded the collagen gel only to a limited extent (FIG. 4B). Taken together, these observations suggest that signaling by the β4 substrate domain promotes endothelial cell migration and invasion, in accordance with the hypothesis that it controls the onset of the invasive phase of angiogenesis.

The β4 substrate domain induces nuclear accumulation of ERK and NF-kB during endothelial cell migration in vitro and angiogenesis in vivo Prior studies had provided evidence that α6β4 controls ERK and NF-kB signaling (Mainiero et al., 1997; Santoro et al., 2003; Zahir et al., 2003). To examine the mechanism by which α6β4 promotes endothelial cell migration, we compared ERK and NF-kB signaling in HUVECs expressing wild-type or mutant α6β4 during migration on laminin-5. The cells were plated at confluency on laminin-5 and, thirty minutes after wounding, subjected to immunofluorescent staining with antibodies to P-ERK and the p65 subunit of NF-kB. The cells expressing wild-type α6β4 displayed significant nuclear accumulation of P-ERK and NF-kB as they entered into the wound. In contrast, those expressing mutant α6β4 did not show-significant nuclear accumulation of P-ERK or NF-kB under the same conditions. These results suggest that α6β4 signaling promotes both ERK and NF-kB signaling in migrating endothelial cells.

To examine the role of ERK and NF-OB signaling in endothelial cell migration, HUVECs expressing wild-type α6β4 were subjected to in vitro wound closure assay in the presence of the MEK inhibitor PD98059 or the NF-kB inhibitor BAYI 1-072. These compounds reduced the migration of α6β4-expressing HUVECs to levels similar to those displayed by control HUVECs or HUVECs expressing mutant α6β4 (FIG. 4A), suggesting that the β4 substrate domain promotes endothelial cell migration by inducing NF-kB and ERK signaling.

To examine if deletion of the β4 substrate domain Impairs signaling in endothelial cells in vivo, sections of Matrigel plugs from wild-type and mutant mice were subjected to double staining with antibodies to PECAM-1 and to P-ERK or the p65 subunit of NF-KB. We observed significant levels of P-ERK and p65 in the nuclei of many endothelial cells of small and intermediate-size vessels from wild-type plugs. By contrast, both signaling molecules were predominantly confined to the cytoplasm in endothelial cells of similar vessels from mutant plugs. To confirm this observation, the samples were subjected to double staining with antibodies to β4 and to P-ERK or p65 and to counterstaining with DAPI. Both P-ERK and p65 accumulated in the nuclei of endothelial cells expressing wild-type β4 but remained largely confined to the cytoplasm in endothelial cells expressing the mutant integrin. Taken together, these results suggest that the β4 substrate domain promotes nuclear translocation of ERK and NF-KB during angiogenesis.

The β4 Substrate Domain Promotes Tumor Angiogenesis

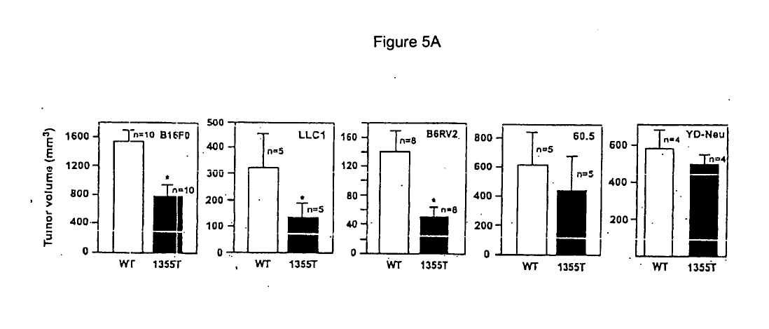

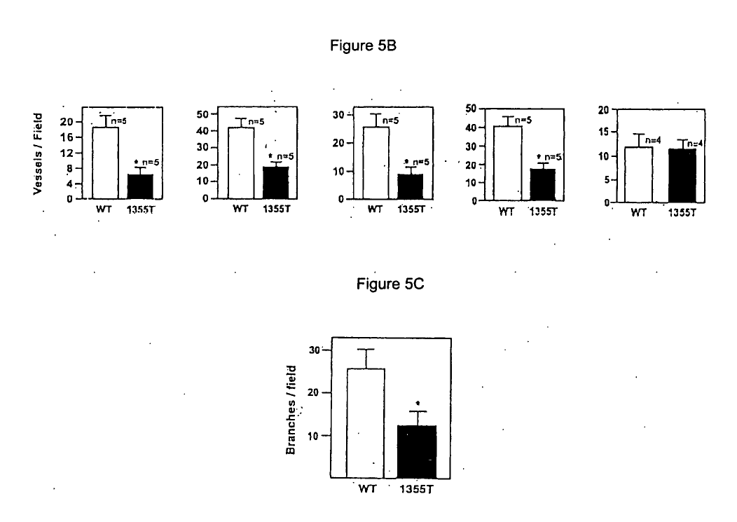

To test the role of the β4 substrate domain in tumor angiogenesis, we injected B16F0 melanoma cells, LLC1 Lewis lung carcinoma cells, B6RV2 lymphoma cells, and 60.5 fibrosarcoma cells s.c. in wild-type and mutant mice. The 816F0, LLC1, and B6RV2 tumors grew in mutant mice to a size significantly smaller than they did in wild-type mice. Although the 60.5 tumors also expanded less rapidly in mutant mice, the reduction in tumor growth was in this case smaller (FIG. 5A). To compare the density of microvessels in the tumors grown in wild-type and mutant mice, we used anti-PECAM-1 staining. The density of microvessels in each of the four tumors grown subcutaneously in mutant mice was significantly reduced as compared to that of tumors grown under identical conditions in wild-type mice (FIG. 5B). This was also true for the 60.5 tumors, which grew relatively well in mutant mice, suggesting that these tumors are somewhat less dependent on angiogenesis for growth. Finally, in the context of other studies, we also examined the effect of loss of β4 signaling on angiogenesis in an orthotopic model of mammary carcinogenesis. In this case, the tumors became vascularized and grew to a similar extent in wild-type and mutant mice (FIGS. 5A and B), suggesting that α6β4 signaling does not contribute to tumor angiogenesis in this specific system. Four major parameters—tumor cell type, transformation mechanism, injection protocol, and specific genetic background of mice—may have influenced the outcome of this specific experiment. Since loss of β4 signaling inhibited tumor angiogenesis to a significant extent in four out of five xenotransplantation models tested, we concluded that α6β4 signaling plays a significant and broad, but perhaps not universal, role in tumor angiogenesis.

To visualize the effect of loss of β4 signaling on tumor vasculature, we injected wild-type and mutant mice bearing 816F0 xenografts with FITC-Lectin. Confocal analysis and 3-D reconstruction confirmed that the defective angiogenic response of mutant mice to tumors was due to reduced branching (FIG. 5C). Two arguments rule out the possibility that immunological factors contribute to the tumor angiogenesis defect of mutant mice. First, α6β4 is not expressed in the immune system. Second, the 60.5 fibrosarcoma, which are derived from 129 Sv mice, were injected in wild-type and mutant mice of pure syngeneic background, making an immunological response unlikely. In addition, the reduced angiogenesis in tumors of mutant mice does not appear to be a consequence of reduced tumor growth, because the 60.5 tumors grew relatively well but evoked reduced angiogenesis in mutant mice. Taken together, these results identify a role for α6β4 signaling in tumor angiogenesis.

The β4 Substrate Domain is not Necessary for Assembly of Hemidesmosomes and Epidermal Adhesion

The skin of mutant newborn mice did not display any gross histological abnormality, except for a slightly decreased thickness. The epidermis appeared tightly attached to the basement membrane and the underlying dermis. No areas of discontinuity were observed at the basement membrane junction, in accordance with the absence of skin fragility in mutant mice. In addition, laminin-5, the mutant integrin, and the hemidesmosomal components HD-1/plectin and BPAG-2 were normally concentrated along the dermal-epidermal junction. Thus, deletion of the C-terminal, signaling domain of b4 does not affect the ability of α6b4 to establish a transmembrane connection between laminin-5 and the hemidesmosomal cytoskeleton and to mediate stable epidermal adhesion in vivo.

Prior studies had indicated that the C-terminal portion of the b4 tail interacts directly with the cytoplasmic domain of the transmembrane collagen Bullous Pemphigoid Antigen-2 (BPAG-2) and thereby with the plakin BPAG-1 (Borradori and Sonnenberg, 1999). Because these interactions are thought to contribute to the assembly of mature hemidesmosomes, we used immunofluorescent staining to examine the ability of mutant keratinocytes to form hemidesmosomes in culture. In agreement with the observation that the C-terminal portion of the b4 tail is necessary for recruitment of BPAG-2 and BPAG-1 to hemidesmosomes (Schaapveld et al., 1998), the mutant keratinocytes assembled in culture hemidesmosome-like adhesions containing HD-1/plectin, but devoid of BPAG-2.

In accordance with the absence of a skin fragility phenotype, transmission electron microscopy (EM) revealed that the skin of mutant mice contained well-structured hemidesmosomes.

Morphometric analysis of the hemidesmosomes of wild-type and mutant mice. indicated that they were similar in number and appearance. (Table 1) The number of hemidesmosomes per cell profile was derived from the photographic reconstruction of 10 cell profiles per group. Morphometric data represent the mean+standard deviation of values obtained from the analysis of 80 hemidesmosomes per group. DBL: distance from basal lamina, HBL: thickness of basal lamina.

TABLE 1

| | Number | | | | |
|---|---|---|---|---|---|
| | (/10 cells) | Height (nm) | Length (nm) | DBL (nm) | HBL (nm) |
| WT | 8.2 ± 3.4 | 36 ± 7 | 155 ± 36 | 36 ± 6 | 34 ± 4 |
| 1355T | 9.5 ± 4.5 | 33 ± 6 | 178 ± 38 | 38 ± 5 | 30 ± 3 |

Furthermore, cryo-immuno EM clearly demonstrated that BPAG-2 is regularly incorporated in the hemidesmosomes of mutant mice. These results provide direct evidence that the N-terminal portion of b4 tail (to amino acid 1355) is sufficient for association with the keratin cytoskeleton and assembly of adhesion-competent hemidesmosomes in vivo.

How do we explain the observation that the C-terminal portion of the b4 tail is required to recruit BPAG-2 to the hemidesmosomes of cultured keratinocytes but not to those of mutant mice in vivo? It is possible that BPAG-2 is recruited to hemidesmosomes through its binding to a basement membrane component of dermal origin. Unable to synthesize this component, cultured keratinocytes would be dependent on the C-terminal segment of b4 tail to recruit BPAG-2 and, hence, BPAG-1 to hemidesmosomes.

Our results provide genetic evidence that the α6β4 integrin promotes tumor angiogenesis—and, presumably, other forms of pathological angiogenesis—by a signaling mechanism. Immunohistochemical and cell biological experiments suggest that α6β4 promotes nuclear translocation of P-ERK and NF-KB and acquisition of an invasive phenotype at the onset of the invasive phase of angiogenesis. These results suggest the intriguing possibility that α6β4 performs a similar function signaling function in cancer cells and in angiogenic endothelial cells.

In order to design effective anti-integrin drugs for anti-angiogenesis, it is important to understand the mechanisms by which specific integrins participate in this process. Prior studies with adhesion-blocking antibodies and RGD-containing peptides have led to the hypothesis that αvβ3 and αvβ5 promote tumor angiogenesis by a signaling mechanism (Eliceiri and Cheresh, 1999). However, genetic studies suggest more complex roles (Hynes, 2002). In particular, it is possible that the αv integrins may have both positive and negative signaling roles during tumor angiogenesis. Perhaps, they stimulate endothelial cell proliferation and migration by binding to components of the interstitial matrix during the invasive phase of tumor angiogenesis, but they induce an active, negative signal at the end of the process, either upon becoming unligated or upon binding to a known negative regulator of angiogenesis, such as Thrombospondin, Tumstatin—a fragment of the c3 chain of type IV collagen, or PEX—a fragment of MMP2 (Sheppard, 2002). In this model, the blocking agents interfere with positive signaling, while allowing negative signaling to occur. Among other integrins involved in angiogenesis, α5β1 has attracted considerable interest. Both knock-out studies and antibody-blocking experiments have indicated that α5β1 and its ligand fibronectin are required for developmental and pathological angiogenesis (Hynes, 2002). However. it is not known at what step of angiogenesis α5β1 functions and whether it acts by an adhesive or signaling mechanism. Our results indicate that α6β4 signaling specifically controls the invasive phase of pathological angiogenesis. In addition to adding to our understanding of integrin function during angiogenesis, these results provide a novel potential target for therapeutic intervention.

The role of α6β4 in angiogenesis described here is unexpected. Prior studies have shown that neither αβ4 nor its signaling functions are required during developmental angiogenesis (Dowling et al., 1996; Murgia et al., 1998; van der Neut et al., 1996). In addition, based on the observation that α6β4 levels increase during vessel maturation, La Flamme and colleagues have proposed that α6β4 limits angiogenesis (Hiran et al., 2003). In retrospect, it is not surprising that α6β4 does not play a role during developmental angiogenesis, as it is expressed in endothelial cells only after completion of this process (Hiran et al., 2003). In addition, our studies do not rule out the possibility that α6β4 also contributes to the maturation of adult vessels. They simply show that that its signaling function contributes to initiate the invasive phase of angiogenesis. We have demonstrated this role of α6β4 signaling in several systems: the Matrigel plug assay, the retinal neovascularization model, and four xenograft models of tumor angiogenesis. This said, increasing evidence indicates that angiogenesis is driven by different growth factors and cytokines and, hence, proceeds by partially distinct mechanisms, depending on developmental stage, tissue, and disease state (LeCouter et al., 2002; Risau, 1997). In particular, the two major angiogenic growth factors, bFGF and VEGF, cooperate with distinct αv integrins to induce angiogenesis (Friedlander et al., 1995; Hood et al., 2003). We have observed that loss of β4 signaling does not suppress angiogenesis in a specific orthotopic model of mammary carcinogenesis. Thus, although our results suggest that α6β4 signaling participates in both bFGF and VEGF-induced angiogenesis, future studies will be necessary to examine how general is the requirement for α6β4signaling during tumor angiogenesis. Since the angiogenic Id transcription factors induce expression of the genes encoding α6β4 and its ligand, laminin-5 (Ruzinova et al., 2003), it is possible that α6β4 signaling is especially important when angiogenesis is driven by Id.

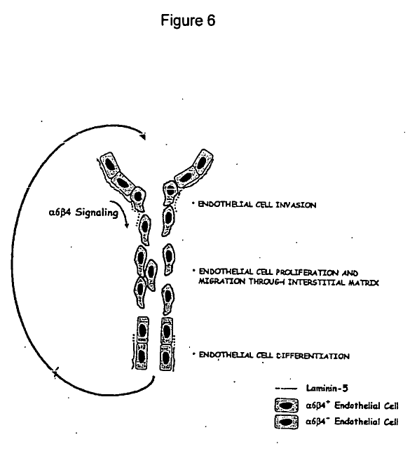

What is the mechanism by which α6β4 signaling controls angiogenesis? α6β4 is expressed during angiogenesis in relatively mature vessels. The endothelial cells of β4+ vessels display a very low proliferative index, making it unlikely that α6β4 signaling promotes endothelial proliferation. In addition. the angiogenic endothelium of mutant mice does not display evidence of increased apoptosis, excluding the hypothesis that α6β4 signaling plays a necessary role in endothelial cell survival. The severe reduction of PECAM+β4-capillaries observed in mutant plugs suggests that α6β4 signaling is necessary for the generation of β4-sprouts from β4+ vessels, i.e. during the initial step of the invasive phase of angiogenesis. As expression of wild-type, but not mutant, α6β4 promotes endothelial cell migration and invasion in vitro, we propose that α6β4 plays a similar role in vivo (FIG. 6). In fact, α6β4 may play a general role during branching morphogenesis, as it has been shown that anti-α6β4 antibodies suppress branching of the ureteric bud in the developing kidney (Zent et al., 2001) and the formation of epithelial cords by breast epithelial cells embedded in Matrigel (Stahl et al., 1997).

Sprouting angiogenesis is thought to commence with the acquisition of an invasive phenotype by specific endothelial cells. The basement membrane underlying these cells is degraded as they migrate into the underlying interstitial matrix. We have observed a defect in nuclear accumulation of ERK and NF-KB in the endothelial cells of small vessels in mutant plugs. This finding suggests that the β4 substrate domain regulates sprouting angiogenesis by promoting nuclear translocation of key transcription factors, and that this event precedes, and it may indeed be necessary for, the acquisition of an invasive phenotype by sprouting endothelial cells. In agreement with this model, prior studies have shown that angiogenesis requires integrin signaling to both ERK and NF-KB (Hood et al., 2003; Klein et al., 2002). Although these transcriptional regulators may play multiple distinct roles in angiogenesis, our observations suggest that they play specific roles in the acquisition of the invasive phenotype. In particular, we have shown that β4 signaling promotes nuclear translocation of P-ERK and NF-KB as endothelial cells commence to migrate on laminin-5 and that these signals are necessary to promote endothelial cell migration in vitro. In agreement with this model, it is known that AP-1 and NF-KB coordinately control the expression of genes involved in cell migration and invasion (Vincenti and Brinckerhoff, 2002).

The possibility of treating chronic diseases, such as diabetic retinopathy, rheumatoid arthritis, and cancer, with anti-angiogenic compounds has attracted considerable interest. Because α6β4 signaling is not required during development and normal adult life, compounds blocking α6β4 signaling may curb pathological angiogenesis without exerting significant toxic effects. In addition, it is clear that neoangiogenesis is an integral component of tumor invasion (Hanahan and Folkman, 1996). As cancer cells invade through the extracellular matrix, they are met by cords of angiogenic endothelial cells, bringing them nourishment. Since α6β4 signaling appears to play key roles in both tumor invasion and tumor angiogenesis, its inhibition may be especially beneficial for cancer therapy.

### EXPERIMENTAL PROCEDURES

### Targeted Deletion of the β4 Substrate Domain

The Cla I/Xba I fragment of mouse β4 gene was isolated from a 129 Sv library (Murgia et al., 1998) and subcloned in pBluescript to generate pB/S-mβ4-Cla I/Xba. Site directed mutagenesis was used to introduce a Nhe I site within the sequences encoding the transmembrane domain of pB/S-mβ4-Cla I/Xba I, as well as pcDNA3-hβ4 (Dans et al., 2001), without altering their reading frames. PCR was used to introduce a stop codon followed by an Xba I and an EcoR I site in pcDNA3-β4, thereby generating pcDNA3-β4 Cyto-1355T. To insert the cDNA fragment encoding the N-terminal portion of the cytoplasmic domain of β4—to amino acid 1355—downstream of and in frame with the exon encoding the transmembrane domain of the protein, a Nhe I/Xba I fragment of pcDNA3-β4 Cyto-1355T was subcloned in pB/Smβ4-Cla I/Xba I and a Cla I/EcoR I fragment of the resulting plasmid was inserted in the targeting vector previously used to delete the entire the cytoplasmic domain of β4 (Murgia et al., 1998). The resulting replacement vector, which carried a left arm of 5 Kb and a right arm of 3.8 Kb, was linearized and electroporated in ES cells. Positively transfected cells which had undergone homologous recombination were selected in 0.5 mg/ml G418 and 0.2 mM Gancyclovir and identified by Southern blotting. Two distinct ES cell lines were found to carry the expected mutation and both were injected into blastocyst-stage C57BL/6 mouse embryos. The embryos were then transplanted into the uteri of pseudopregnant C57BL/6 mice. Extensively chimeric mice derived from both lines were crossed to C57BL/6 females. Heterozygous offspring were used to generate mice homozygous for the targeted deletion. Mice were genotyped by PCR using tail genomic DNA. The following primers were used for amplification: 5′-ggaaatagcagagcaggatac-3′ (wild-type) (Seq. ID No. 7), 5′-ctcgtgcmacggtatcgc-3′ (recombinant) (Seq. ID No. 8), 5′-ctcggttgcagcaaggaag-3′ (common) (Seq. ID No. 9). For Southern blotting, tail genomic DNA was digested with Nco I and, after agarose gel electrophoresis and transfer to a nylon membrane, hybridized to a 500 bp radioactive cDNA probe complementary to sequences in the extracellular domain of β4, as described previously (Murgia et al., 1998). Except when indicated, the experiments were conducted on mice of mixed genetic background.

### Cells, Antibodies, and Other Reagents

Keratinocytes were isolated from the skin of newborn mice and grown on collagen I-coated plates in EMEM.06 with 8% Chelex-treated FBS, 2 ng/ml EGF, and 0.06 mM CaCl₂ (Hager et al., 1999). Primary HUVECs were cultured on gelatin-coated dishes (Klein et al., 2002). Rat mAbs to β4 (346-11A), α6 (GoH3), and PECAM-1 (MEC 13.3) were from Pharmingen. Goat anti-PECAM-1 (M-20) and anti-β4 (C-20) and rabbit anti-VEGF-R2 (C-20) and -NF-KB p65 (C-20) were from Santa Cruz. Rabbit anti-P-ERK and anti-P-AKT were from Cell Signaling and anti-keratin-5 (AF 138) from Babco. Mouse mAbs to smooth muscle α-actin (clone 1 A4) and to 0-actin (clone AC-74) were from Sigma and to NF-KB p65 (clone 2A12A7) from Zymed. Affinity-purified rabbit antibodies to the LE4-6 modules of the mouse laminin y2 chain (Sasaki et al., 2001), mouse mAb 3E1 to β4 and rabbit anti-β4-exo serum to a GST fusion protein comprising the N-terminal domain of β4 (Mainiero et al., 1997) were described previously. FITC- and Cy3-conjugated affinity-purified secondary antibodies were from Jackson Laboratories. Laminin 5 matrices were prepared as previously described (Spinardi et al., 1995). Purified laminin-5 was from Chemicon. Human fibronectin and rat tail collagen type I were from Collaborative Research. FITC-Lectin (isolectin B4) was from Vector Laboratories. The MEK inhibitor PD98059 and the NF-KB inhibitor BAY 11-7082 were from Calbiochem.

Keratinocyte Studies

For immunoprecipitation and immunoblotting analyses, keratinocytes were lysed in RIPA buffer with 10 mM EDTA and protease inhibitors. Equivalent amounts of total proteins were immunoprecipitated with mAb GoH3 and subjected to immunoblotting with anti-β4-exo or directly subjected to immunoblotting. FACS analysis and adhesion assays were performed as previously described (Murgia et al., 1998). For signaling studies, the keratinocytes were plated on laminin-5 or collagen I for the indicated times, lysed, and subjected to immunoblotting with anti-phospho-ERK and anti-phospho-AKT.

Endothelial Cell Studies

HUVECs were electroporated with equimolar amounts of plasmids encoding α6 and either β4 or β4-1355T (Dans et al., 2001), deprived of growth factors for 18 hours, and then panned on plates coated with the anti-β4 mAb 3E1. Bound cells were washed with PBS and recovered by trypsin-EDTA treatment. For in vitro wound assays, equal numbers of cells expressing wild-type β4 or mutant β4-1355T were plated on dishes coated with laminin-5, grown until confluent, and starved. Monolayers were scratched with a P200 pipette tip and incubated in the presence of serum and 20 ng/ml bFGF for 18 hours. Wound closure was monitored by digital photography. To monitor ERK and NF-KB signaling during migration, control and transfected HUVECs were subjected to In vitro wounding for 30 minutes, fixed with 3.7% formaldehyde, and subjected to immunofluorescent staining with anti-P-ERK and anti-p65, as described (Klein et al., 2002). To examine the effect of β4 signaling on endothelial cell invasion, control and transfected HUVECs were grown on Biosolin Cytodex-3 microcarrier beads (NUNC) until confluent. The beads were then placed in collagen gels (3D Collagen Cell Culture Kit, Chemicon). The gels were overlaid with DMEM with with 10% fetal bovine serum, 2 mmol glutamine and 10 ng/ml bFGF. HUVECS Invasion was quantified 72 hours later by counting the average number of capillary-like structures per microcarrier bead.

Immunofluorescence Microscopy and Immunohistochemistry

Tissues and plugs were embedded in paraffin or snap-frozen in OCT compound (Tissue-Tek). Paraffin-embedded sections were stained with Hematoxylin and Eosin or subjected to Immunoperoxidase staining with the indicated antibodies using the ABC Staining Kit (Vector Laboratories). Frozen sections were subjected to Immunofluorescent staining with the indicated antibodies. To measure cell proliferation in vivo, mice were injected i.v. with 5 μM BrdU/100 g body weight and sacrificed 1 hour later. Cryostat as well as paraffin-embedded sections of matrigel or tumors were subjected to immunofluorescent or immunohistochemical staining with anti-BrdU antibodies (BrdU Labeling and Detection Kit 1, Roche). To estimate cell death in vivo, TUNEL assays were performed on paraffin-embedded sections (In Situ Cell Death Detection Kit, Roche).

Matrigel Plug Assay

Eight week-old mice were injected s.c. with 400 □l of growth factor-depleted Matrigel (BD Biosciences) supplemented with 400 ng/ml bFGF and 1 □g/ml heparin sulphate and sacrificed 7 days later (Passaniti et al., 1992). To visualize angiogenesis, the mice were injected intravenously with 20 μg of FrTC-isolectin B4 (Vector Labs) 30 minutes before harvesting the plugs. Fluorescently labeled vessels were examined by confocal microscopy. To quantify angiogenesis, FITC-Lectin containing plugs were homogenized in RIPA buffer containing protease and phosphatase inhibitors and subjected to fluorimetric analysis. Alternatively, plug lysates were immunoprecipitated and subjected to immunoblotting with anti-VEGF-R2 antibodies. Each experimental group consisted of five mice. Each mouse was injected with Matrigel alone or Matrigel supplemented with bFGF and heparin sulphate. Experiments were repeated three times.

Retinal Hypoxia Model

P7 mice were exposed to 75% oxygen for 5 days and then returned to normoxic conditions for 5 days. Mice of the same age kept in normal air were used as controls. Eyes were fixed in 4% paraformaldehyde, embedded in paraffin, sectioned and subjected to staining. Angiogenesis was quantified by counting the number of PECAM-1 positive glomeruli penetrating the inner limiting membrane.

Tumor Xenografts

Six month-old mice were injected s.c. with 10⁶ tumor cells per flank. B6RV2 human lymphoma cells, B16F0 mouse melanoma cells, and LLC1 Lewis Lung carcinoma cells were injected in wild-type and mutant mice of mixed genetic background. The 60.5 fibrosarcoma cells, which are derived from 129 Sv mice (Pozzi et al., 2000), were injected in syngeneic wild-type and mutant mice of pure background. The YD-Neu mouse mammary carcinoma cells—generated by introducing rat Neu In YD cells (Dankort et al., 2001)—were implanted orthotopically at 5×10⁶ in Matrigel diluted 1:1 in PBS. To avoid an immune response to rat Neu, the cells were injected in MMTV-Neu transgenic mice expressing either wild-type or mutant β4, as these mice are tolerant to rat Neu. These mice had been backcrossed into an FVB/n background. The tumors were excised after the indicated number of days. Final tumor dimensions were measured by caliper.

Immunofluorescence Microscopy and Immunohistochemistry:

For immunofluorescent detection of hemidesmosome-like adhesions, keratinocytes were permeabilized/extracted with 0.2% Triton X-100 and fixed with cold methanol as described previously (Dans et al., 2001). They were then stained with the indicated antibodies.

Electron Microscopy and Immunogold Labeling:

Skin samples were collected, fixed with 2.5% glutaraldehyde in 0.1 M cacodylate buffer pH 7.2, post fixed with 1% osmium tetroxide, stained en bloc with 1% uranyl-acetate, and embedded in Polybed 812. Ultra-thin sections were stained with uranyl acetate and lead citrate and examined with a ZEISS EM 902 or a Philips CM10 electron microscope. Immunogold, labeling was performed on ultra-thin cryosections as previously described (Liou et al., 1996).

### REFERENCES

The following references are cited herein. These and the other references cited herein are incorporated herein by reference in their entirety.
Arbeit, J. M., Munger, K., Howley, P. M., and Hanahan, D. (1994). Progressive squamous epithelial neoplasia in K14-HPV16 transgenic mice. J. Virol. 68, 4358-4368.
Arbeit, J. M., Olson, D. C., and Hanahan, D. (1996). Upregulation of FGFs and their receptors during multi-stage epidermal carcinogenesis in K14-HPV16 transgenic mice. Oncogene 13,1847-1857.
Bergers, C., Brekken, R., McMahon, C., Vu, T. H., Itoh, T., Tamaki, K., Tanzawa, K., Thorpe, P., Itohara, S., Werb, Z., and Hanahan, D. (2000). MMP-9 triggers the angiogenic switch during carcinogenesis. Nat. Cell Biol. 2, 737-744.
Byzova, T. V., Goldman, C. K., Pampori, N., Thomas, K. A., Bett, A., Shattil, S. J., and Plow, E. F. (2000). A mechanism for modulation of cellular responses to VECF: activation of the integrins. Mol. Cell 6, 851-860.
Coussens, L. M., Tinkle, C. L., Hanahan, D., and Werb, Z. (2000). MMP-9 supplied by bone marrow-derived cells contributes to skin carcinogenesis. Cell 103, 481-490.
Dankort, D., Maslikowski, B., Warner, N., Kanno, N., Kim, H., Wang, Z., Moran, M. F., Oshima, R. G., Cardiff, R. D., and Muller, W J. (2001). Grb2 and Shc adapter proteins play distinct roles in Neu (ErbB2)-induced mammary tumorigenesis: implications for human breast cancer. Mol. Cell. Biol. 21, 1540-1551.
Dans, M., Gagnoux-Palacios. L., Blaikie, P., Klein, S., Mariotti, A., and Giancotti, F. G. (2001). Tyrosine phosphorylation of the b4 integrin cytoplasmic domain mediates Shc signaling to ERK and antagonizes formation of hemidesmosomes. J. Biol. Chem. 276, 1494-1502.
Dowling, J, Yu, Q. C., and Fuchs, E. (1996). b4 integrin is required for hemidesmosome formation, cell adhesion and cell survival. J. Cell Biol./34, 559-572.
Einheber, S., Milner, T. A., Glancotti, F., and Salzer, J. L. (1993). Axonal regulation of Schwann cell integrin expression suggests a role for α6b4 in myelination. J. Cell Biol. 723,1223-1236.
Eliceiri; B. P., and Cheresh, D. A. (1999). The role of av integrins during angiogenesis: insights into potential mechanisms of action and clinical development. J Clin. Invest. 103, 1227-1230.
Folkman, J., Watson, K., Ingber, D., and Hanahan, D. (1989). Induction of angiogenesis during the transition from hyperplasia to neoplasia. Nature 339, 58-61.
Friedlander, M., Brooks, P. C., Shaffer, R. W., Kincaid, C. M., Varner, J. A., and Cheresh, D. A. (1995). Definition of two angiogenic pathways by distinct av integrins. Science 270, 1500-1502.
Gagnoux-Palacios, L., Dans, M., van't Hof, W., Mariotti, A., Pepe, A., Meneguzzi, G., Resh, M. D., and Giancotti, F. G. (2003). Compartmentalization of integrin a6b4 signaling in lipid rafts. J. Cell Biol./62, 1189-1196.
Gambaletta, D., Marchetti, A., Benedetti, L., Mercurio, A. M., Sacchi, A., and Falcioni, R. (2000). Cooperative signaling between a6b4 integrin and ErbB-2 receptor is required to promote PI-3K-dependent invasion. J. Biol. Chem. 275, 10604-10610.
Giancotti, F. G., and Ruoslahti, E. (1999). Integrin signaling. Science 285, 1028-1032.
Giancotti, F. G., and Tarone, G. (2003). Positional control of cell fate through joint integrin-receptor protein tyrosine kinase signaling. Annu. Rev. Cell Dev. Biol. 79,173-206.
Hager, B., Bickenbach, J. R., and Fleckman, P. (1999). Long-term culture of murine epidermal keratinocytes. J. Invest. Dermatol. 112, 971-976.
Hanahan, D. (1985). Heritable formation of pancreatic beta-cell tumours in transgenic mice expressing recombinant insulin/simian virus 40 oncogenes. Nature 315, 115-122.
Hanahan, D., and Folkman, J. (1996). Patterns and emerging mechanisms of the angiogenic switch during tumorigenesis. Cell 86, 353-364.
Hiran, T. S., Mazurkiewicz, J. E., Kreienberg, P., Rice, F. L., and LaFlamme, S. E. (2003). Endothelial expression of the a6b4 integrin is negatively regulated during angiogenesis. J. Cell Sci. 116, 3771-3781.
Holash, J., Maisonpierre, P. C., Compton, D., Boland, P., Alexander, C. R., Zagzag, D., Yancopoulos, G. D., and Wiegand, S. J. (1999). Vessel cooption, regression, and growth in tumors mediated by angiopoietins and VEGF. Science 284, 1994-1998.
Hood, J. D., Frausto, R., Kiosses, W. B., Schwartz, M. A., and Cheresh, D. A. (2003). Differential av integrin-mediated Ras-ERK signaling during two pathways of angiogenesis. J. Cell Biol. 162, 933-943.

Hynes, R. O. (2002). A reevaluation of integrins as regulators of angiogenesis. Nat. Med. 8, 918-921.
Inoue, M., Hager, J. H., Ferrara, N., Gerber, H. P., and Hanahan, D. (2002). VEGF-A has a critical, nonredundant role in angiogenic switching and pancreatic beta cell carcinogenesis. Cancer Cell 1, 193-202.
Klein, S., de Fougerolles, A. R., Blaikie, P., Khan, L., Pepe, A., Green, C. D., Koteliansky, V., and Giancotti, F. G. (2002). a5b1 integrin activates an NF-kB-dependent program of gene expression important for angiogenesis and inflammation. Mol. Cell. Biol. 22, 5912-5922.
Lal, K. M., and Pawson, T. (2000). The ShcA phosphotyrosine docking protein sensitizes cardiovascular signaling in the mouse embryo. Genes Dev. 14, 1132-1145.
LeCouter, J., Lin, R., and Ferrara, N. (2002). Endocrine gland-derived VEGF and the emerging hypothesis of organ-specific regulation of angiogenesis. Nature Med. 8, 913-917.
Mainiero, F., Murgia, C., Wary, K. K., Curatola, A. M., Pepe, A., Blumemberg, M., Westwick, J. K., Der, C. J., and Ciancotti, F. G. (1997). The coupling of a6b4 integrin to Ras-MAP kinase pathways mediated by Shc controls keratinocyte proliferation. EMBO J. 16, 2365-2375.
Mainiero, F., Pepe, A., Wary, K. K., Spinardi, L., Mohammadi, M., Schlessinger, J., and Giancotti, F. G. (1995). Signal transduction by the a6b4 integrin: distinct b4 subunit sites mediate recruitment of Shc/Grb2 and association with the cytoskeleton of hemidesmosomes. EMBO J. 14, 4470-4481.
Miranti, C. K., and Brugge, J. S. (2002). Sensing the environment: a historical perspective on integrin signal transduction. Nat. Cell Biol. 4, E83-90.
Murgia, C., Blaikie, P., Kim, N., Dans, M., Petrie, H. T., and Giancotti, F. G. (1998). Cell cycle and adhesion defects in mice carrying a targeted deletion of the integrin b4 cytoplasmic domain. EMBO J. 17,3940-3951.
Passaniti, A., Taylor, R. M., Pili, R., Guo, Y., Long, P. V., Haney, J. A., Pauly, R. R., Grant, D. S., and Martin, C. R. (1992). A simple, quantitative method for assessing angiogenesis and antiangiogenic agents using reconstituted basement membrane, heparin, and fibroblast growth factor. Lab Invest. 67, 519-528.
Pozzi, A., Moberg, P. E., Miles, L. A., Wagner, S., Soloway, P., and Gardner, H. A. (2000). Elevated matrix metalloprotease and angiostatin levels in integrin al knockout mice cause reduced tumor vascularization. Proc. Natl. Acad. Sci. USA 97, 2202-2207.
Risau, W. (1997). Mechanisms of angiogenesis. Nature 386, 671-674.
Ruzinova, M. B., Schoer, R. A., Gerald, W., Egan, J. E., Pandolfi, P. P., Rafli, S., Manova, K., Mittal, V., and Benezra, R. (2003). Effect of angiogenesis inhibition by Id loss and the contribution of bone-marrow-derived endothelial cells in spontaneous murine tumors. Cancer Cell 4, 277-289.
Santoro, M. M., Gaudino, G., and Marchisio, P. C. (2003). The MSP receptor regulates a6b4 and a3b1 integrins via 14-3-3 proteins in keratinocyte migration. Dev. Cell 5, 257-271.
Sasaki, T., Gohring, W., Mann, K., Brakebusch, C., Yamada, Y., Fassler, R., and Timpl, R. (2001). Short arm region of laminin-5 g2 chain: structure, mechanism of processing and binding to heparin and proteins. J. Mol. Biol. 314, 751-763.
Schaapveld, R. Q., Borradori, L., Geerts, D., van Leusden, M. R., Kuikman, I., Nievers, M. G., Niessen, C. M., Steenbergen, R. D., Snijders, P. J., and Sonnenberg, A. (1998). Hemidesmosome formation is initiated by the b4 integrin subunit, requires complex formation of b4 and HD1/plectin, and involves a direct interaction between b4 and the bullous pemphigoid antigen 180. J. Cell Biol. 142, 271-284.
Seifert, P., and Spitznas, M. (2002). Axons in human choroidal melanoma suggest the participation of nerves in the control of these tumors. Am. J. Ophthalmol. 133, 711-713.
Serini, G., Valdembri, D., Zanivan, S., Morterra, G., Burkhardt, C., Caccavari, F., Zammataro, L., Primo, L., Tamagnone, L., Logan, M., Tessier-Lavigne, M., Taniguchi, M., Puschel, A. W., and Bussolino, F. (2003). Class 3 semaphorins control vascular morphogenesis by inhibiting integrin function. Nature 424, 391-397.
Shaw, L. M., Rabinovitz, I., Wang, H. H., Toker, A., and Mercurio, A. M. (1997). Activation of PI-3K by the a6b4 integrin promotes carcinoma invasion. Cell 97, 949-960.
Sheppard, D. (2002). Endothelial integrins and angiogenesis: not so simple anymore. J. Clin. Invest. .110, 913-914.
Shweiki, D., Itin, A., Soffer, D., and Keshet, E. (1992). Vascular endothelial growth factor induced by hypoxia may mediate hypoxia-initiated angiogenesis. Nature 359, 843-845.
Spinardi, L., Einheber, S., Cullen, T., Milner, T. A., and Giancotti, F. G. (1995). A recombinant tail-less integrin b4 subunit disrupts hemidesmosomes but does not suppress a6b4-mediated cell adhesion to laminins. J. Cell Biol. 729, 473-487.
Spinardi, L, Ren, Y. L., Sanders, R., and Giancotti, F. G. (1993). The b4 subunit cytoplasmic domain mediates the interaction of a6b4 integrin with the cytoskeleton of hemidesmosomes. Mol. Biol. Cell 4, 871-884.
Stahl, S., Weitzman, S., and Jones, J. C. (1997). The role of laminin-5 and its receptors in mammary epithelial cell branching morphogenesis. J. Cell Sci. 170, 55-63.
Tan, C., Cruet-Hennequart, S., Troussard, A., Fazi, L., Costello, P., Sutton, K., Wheeler, J., Gleave, M., Sanghera, J., and Dedhar, S. (2004). Regulation of tumor angiogenesis by integrin-linked kinase (ILK). Cancer Cell 5, 79-90.
Trusolino, L., Bertotti, A., and Comoglio, P. M. (2001). A signaling adapter function for a6b4 integrin in the control of HGF-dependent invasive growth. Cell 107, 643-654.
van der Neut, R., Krimpenfort, P., Calafat, J., Niessen, C. M., and Sonnenberg, A. (1996). Epithelial detachment due to absence of hemidesmosomes in integrin b4 null mice. Nat. Genet. 13, 366-369.
Vincenti, M. P., and Brinckerhoff, C. E. (2002). Transcriptional regulation of collagenase (MMP-1, MMP-13) genes in arthritis: integration of complex signaling pathways for the recruitment of gene-specific transcription factors. Arthritis Res. 4, 157-164.
Zahir, N., Lakins, J. N., Russell, A., Ming, W., Chatterjee, C., Rozenberg, G. I., Marinkovich, M. P., and Weaver, V. M. (2003). Autocrine laminin-5 ligates a6b4 integrin and activates RAC and NFkB to mediate anchorage-independent survival of mammary tumors. J. Cell Biol. 163, 1397-1407.
Zent, R., Bush, K. T., Pohl, M. L., Quaranta, V., Koshikawa, N., Wang, Z., Kreldberg, J. A., Sakurai, H., Stuart, R. O., and Nigam, S. K. (2001). involvement of laminin binding integrins and laminin-5 in branching morphogenesis of the ureteric bud during kidney development. Dev. Biol. 238, 289-302.
Geiger, B., Bershadsky, A., Pankov, R., and Yamada, K. M. (2001). Transmembrane crosstalk between the extracellular matrix and the cytoskeleton. Nat. Rev. Mol. Cell Biol. 2, 793-805.
Hynes, R. O. (2003). Metastatic potential: generic predisposition of the primary tumor or rare, metastatic variants—or both? Cell 113 821-823.
Klein, S., Giancotti, F. C., Presta, M., Albelda, S. M., Buck, C. A., and Rifkin, D. S. (1993). Basic fibroblast growth factor modulates integrin expression in microvascular endothelial cells. Mol. Biol. Cell 4, 973-982.
Mariotti, A., Kedeshian, P. A., Dans, M., Curatola, A. M., Gagnoux-Palacios, L., and Ciancotti, F. G. (2001). EGF-R signaling through Fyn kinase disrupts the function of integrin a6b4 at hemidesmosomes: role in epithelial cell migration and carcinoma Invasion. J. Cell Biol. 155, 447-457.
Mercurio, A. M., and Rabinovitz, I. (2001). Towards a mechanistic understanding of tumor invasion—lessons from the a6b4 integrin. Semin. Cancer Biol. 11, 129-141.
Schlaepfer, D. D., and Hunter, T. (1998). Integrin signalling and tyrosine phosphorylation: just the FAKs? Trends Cell Biol. 8, 151-157.
Shaw, L. M. (2001). Identification of insulin Receptor Substrate 1 (IRS-1) and IRS-2 as Signaling Intermediates in the α6b4 Integrin-Dependent Activation of P13-K and Promotion of Invasion. Mol. Cell Biol. 21, 5082-5093.
Wary, K. K., Mariotti, A., Zurzolo, C., and Giancotti, F. G. (1998). A requirement for caveolin-1 and associated kinase Fyn in integrin signaling and anchorage-dependent cell growth. Cell 94, 625-634.
Weaver, V. M., Lelievre, S., Lakins, J. N., Chrenek, M. A., Jones, J. C., Ciancotti, F., Werb, Z., and Bissell, M. J. (2002). b4 integrin-dependent formation of polarized three-dimensional architecture confers resistance to apoptosis in normal and malignant mammary epithelium. Cancer Cell 2, 205-216.
Xia, Y., Gil, S. G., and Carter, W. G. (1996). Anchorage mediated by integrin a6b4 to laminin 5 (epiligrin) regulates tyrosine phosphorylation of a membrane-associated 80-kD protein. J. Cell Biol. 132, 727-740.

1. A method for inhibition of angiogenesis in a tissue expressing α6β4 integrin, comprising the steps of exposing the tissue to a therapeutic agent effective to reduce the amount of active α6β4 integrin in the tissue, wherein the therapeutic agent targets β4.

2. The method of claim 1, wherein the tissue is present in a living organism.

3. The method of claim 2, wherein the living organism is human.

4. The method of claim 1, wherein the angiogenesis to be inhibited is pathological angiogenesis.

5. The method of claim 4, wherein the therapeutic agent is an antibody.

6. The method of claim 4, wherein the therapeutic agent is an RNAi species.

7. A method for treatment of a disease condition associated with pathological angiogenesis in a patient, comprising the stop of administering to the patient an amount of a therapeutic agent effective to reduce the amount of active α6β4 integrin, wherein the therapeutic agent targets Γ4.

8. The method of claim 7, wherein the patient is human.

9. The method of claim 8, wherein the therapeutic agent is an antibody.

10. The method of claim 8, wherein the therapeutic agent is an RNAi species.

11-14. (canceled)

15. The method of claim 7, wherein the therapeutic agent is an antibody.

16. The method of claim 7, wherein the therapeutic agent is an RNAi species.

17. The method of claim 3, wherein the therapeutic agent is an antibody.

18. The method of claim 3, wherein the therapeutic agent is an RNAi species.

19. The method of claim 1, wherein the therapeutic agent is an antibody.

20. The method of claim 1, wherein the therapeutic agent is an RNAi species.


SEQUENCE LISTING (9 sequences)

SEQ ID NO: 1 - protein, 245 aa, artificial
  human integrin-beta-4 binding protein
MAVRASFENNCEIGCFAKLTNTYCLVAIGGSENFYSVFEGELSDTIPVVHASIAGCRIIGRMCVGNRHGLLVPNNTTDQELQHIRNSLPDTVQIRRVEERLSALGNVTTCNDYVALVHPDLDRETEEILADVLKVEVFRQTVADQVLVGSYCVFSNQGGLVHPKTSIEDQDELSSLLQVPLVAGTVNRGSEVIAAGMVVNDWCAFCGLDTTSTELSVVESVFKLNEAQPSTIATSMRDSLIDSLT

SEQ ID NO: 2 - DNA, 5469 bp, human
atggcagggccacgccccagcccatgggccaggctgctcctggcagccttgatcagcgtcagcctctctgggaccttggcaaaccgctgcaagaaggccccagtgaagagctgcacggagtgtgtccgtgtggataaggactgcgcctactgcacagacgagatgttcagggaccggcgctgcaacacccaggcggagctgctggccgcgggctgccagcgggagagcatcgtggtcatggagagcagcttccaaatcacagaggagacccagattgacaccaccctgcggcgcagccagatgtccccccaaggcctgcgggtccgtctgcggcccggtgaggagcggcattttgagctggaggtgtttgagccactggagagccccgtggacctgtacatcctcatggacttctccaactccatgtccgatgatctggacaacctcaagaagatggggcagaacctggctcgggtcctgagccagctcaccagcgactacactattggatttggcaagtttgtggacaaagtcagcgtcccgcagacggacatgaggcctgagaagctgaaggagccctggcccaacagtgacccccccttctccttcaagaacgtcatcagcctgacagaagatgtggatgagttccggaataaactgcagggagagcggatctcaggcaacctggatgctcctgagggcggcttcgatgccatcctgcagacagctgtgtgcacgagggacattggctggcgcccggacagcacccacctgctggtcttctccaccgagtcagccttccactatgaggctgatggcgccaacgtgctggctggcatcatgagccgcaacgatgaacggtgccacctggacaccacgggcacctacacccagtacaggacacaggactacccgtcggtgcccaccctggtgcgcctgctcgccaagcacaacatcatccccatctttgctgtcaccaactactcctatagctactacgagaagcttcacacctatttccctgtctcctcactgggggtgctgcaggaggactcgtccaacatcgtggagctgctggaggaggccttcaatcggatccgctccaacctggacatccgggccctagacagcccccgaggccttcggacagaggtcacctccaagatgttccagaagacgaggactgggtcctttcacatccggcggggggaagtgggtatataccaggtgcagctgcgggcccttgagcacgtggatgggacgcacgtgtgccagctgccggaggaccagaagggcaacatccatctgaaaccttccttctccgacggcctcaagatggacgcgggcatcatctgtgatgtgtgcacctgcgagctgcaaaaagaggtgcggtcagctcgctgcagcttcaacggagacttcgtgtgcggacagtgtgtgtgcagcgagggctggagtggccagacctgcaactgctccaccggctctctgagtgacattcagccctgcctgcgggagggcgaggacaagccgtgctccggccgtggggagtgccagtgcgggcactgtgtgtgctacggcgaaggccgctacgagggtcagttctgcgagtatgacaacttccagtgtccccgcacttccgggttcctgtgcaatgaccgaggacgctgctccatgggccagtgtgtgtgtgagcctggttggacaggcccaagctgtgactgtcccctcagcaatgccacctgcatcgacagcaatgggggcatctgtaatggacgtggccactgtgagtgtggccgctgccactgccaccagcagtcgctctacacggacaccatctgcgagatcaactactcggcgatccacccgggcctctgcgaggacctacgctcctgcgtgcagtgccaggcgtggggcaccggcgagaagaaggggcgcacgtgtgaggaatgcaacttcaaggtcaagatggtggacgagcttaagagagccgaggaggtggtggtgcgctgctccttccgggacgaggatgacgactgcacctacagctacaccatggaaggtgacggcgcccctgggcccaacagcactgtcctggtgcacaagaagaaggactgccctccgggctccttctggtggctcatccccctgctcctcctcctcctgccgctcctggccctgctactgctgctatgctggaagtactgtgcctgctgcaaggcctgcctggcacttctcccgtgctgcaaccgaggtcacatggtgggctttaaggaagaccactacatgctgcgggagaacctgatggcctctgaccacttggacacgcccatgctgcgcagcgggaacctcaagggccgtgacgtggtccgctggaaggtcaccaacaacatgcagcggcctggctttgccactcatgccgccagcatcaaccccacagagctggtgccctacgggctgtccttgcgcctggcccgcctttgcaccgagaacctgctgaagcctgacactcgggagtgcgcccagctgcgccaggaggtggaggagaacctgaacgaggtctacaggcagatctccggtgtacacaagctccagcagaccaagttccggcagcagcccaatgccgggaaaaagcaagaccacaccattgtggacacagtgctgatggcgccccgctcggccaagccggccctgctgaagcttacagagaagcaggtggaacagagggccttccacgacctcaaggtggcccccggctactacaccctcactgcagaccaggacgcccggggcatggtggagttccaggagggcgtggagctggtggacgtacgggtgcccctctttatccggcctgaggatgacgacgagaagcagctgctggtggaggccatcgacgtgcccgcaggcactgccaccctcggccgccgcctggtaaacatcaccatcatcaaggagcaagccagagacgtggtgtcctttgagcagcctgagttctcggtcagccgcggggaccaggtggcccgcatccctgtcatccggcgtgtcctggacggcgggaagtcccaggtctcctaccgcacacaggatggcaccgcgcagggcaaccgggactacatccccgtggagggtgagctgctgttccagcctggggaggcctggaaagagctgcaggtgaagctcctggagctgcaagaagttgactccctcctgcggggccgccaggtccgccgtttccacgtccagctcagcaaccctaagtttggggcccacctgggccagccccactccaccaccatcatcatcagggacccagatgaactggaccggagcttcacgagtcagatgttgtcatcacagccaccccctcacggcgacctgggcgccccgcagaaccccaatgctaaggccgctgggtccaggaagatccatttcaactggctgcccccttctggcaagccaatggggtacagggtaaagtactggattcagggcgactccgaatccgaagcccacctgctcgacagcaaggtgccctcagtggagctcaccaacctgtacccgtattgcgactatgagatgaaggtgtgcgcctacggggctcagggcgagggaccctacagctccctggtgtcctgccgcacccaccaggaagtgcccagcgagccagggcgtctggccttcaatgtcgtctcctccacggtgacccagctgagctgggctgagccggctgagaccaacggtgagatcacagcctacgaggtctgctatggcctggtcaacgatgacaaccgacctattgggcccatgaagaaagtgctggttgacaaccctaagaaccggatgctgcttattgagaaccttcgggagtcccagccctaccgctacacggtgaaggcgcgcaacggggccggctgggggcctgagcgggaggccatcatcaacctggccacccagcccaagaggcccatgtccatccccatcatccctgacatccctatcgtggacgcccagagcggggaggactacgacagcttccttatgtacagcgatgacgttctacgctctccatcgggcagccagaggcccagcgtctccgatgacactggctgcggctggaagttcgagcccctgctgggggaggagctggacctgcggcgcgtcacgtggcggctgcccccggagctcatcccgcgcctgtcggccagcagcgggcgctcctccgacgccgaggcccccacggccccccggacgacggcggcgcgggcgggaagggcggcagccgtgccccgcagtgcgacacccgggccccccggagagcacctggtgaatggccggatggactttgccttcccgggcagcaccaactccctgcacaggatgaccacgaccagtgctgctgcctatggcacccacctgagcccacacgtgccccaccgcgtgctaagcacatcctccaccctcacacgggactacaactcactgacccgctcagaacactcacactcgaccacactgcccagggactactccaccctcacctccgtctcctcccacgactctcgcctgactgctggtgtgcccgacacgcccacccgcctggtgttctctgccctggggcccacatctctcagagtgagctggcaggagccgcggtgcgagcggccgctgcagggctacagtgtggagtaccagctgctgaacggcggtgagctgcatcggctcaacatccccaaccctgcccagacctcggtggtggtggaagacctcctgcccaaccactcctacgtgttccgcgtgcgggcccagagccaggaaggctggggccgagagcgtgagggtgtcatcaccattgaatcccaggtgcacccgcagagcccactgtgtcccctgccaggctccgccttcactttgagcactcccagtgccccaggcccgctggtgttcactgccctgagcccagactcgctgcagctgagctgggagcggccacggaggcccaatggggatatcgtcggctacctggtgacctgtgagatggcccaaggaggagggccagccaccgcattccgggtggatggagacagccccgagagccggctgaccgtgccgggcctcagcgagaacgtgccctacaagttcaaggtgcaggccaggaccactgagggcttcgggccagagcgcgagggcatcatcaccatagagtcccaggatggaggacccttcccgcagctgggcagccgtgccgggctcttccagcacccgctgcaaagcgagtacagcagcatcaccaccacccacaccagcgccaccgagcccttcctagtggatgggctgaccctgggggcccagcacctggaggcaggcggctccctcacccggcatgtgacccaggagtttgtgagccggacactgaccaccagcggaacccttagcacccacatggaccaacagttcttccaaacttga

SEQ ID NO: 3 - DNA, 3222 bp, human
atggccgccgccgggcagctgtgcttgctctacctgtcggcggggctcctgtcccggctcggcgcagccttcaacttggacactcgggaggacaacgtgatccggaaatatggagaccccgggagcctcttcggcttctcgctggccatgcactggcaactgcagcccgaggacaagcggctgttgctcgtgggggccccgcgcggagaagcgcttccactgcagagagccaacagaacgggagggctgtacagctgcgacatcaccgcccgggggccatgcacgcggatcgagtttgataacgatgctgaccccacgtcagaaagcaaggaagatcagtggatgggggtcaccgtccagagccaaggtccagggggcaaggtcgtgacatgtgctcaccgatatgaaaaaaggcagcatgttaatacgaagcaggaatcccgagacatctttgggcggtgttatgtcctgagtcagaatctcaggattgaagacgatatggatgggggagattggagcttttgtgatgggcgattgagaggccatgagaaatttggctcttgccagcaaggtgtagcagctacttttactaaagactttcattacattgtatttggagccccgggtacttataactggaaagggattgttcgtgtagagcaaaagaataacactttttttgacatgaacatctttgaagatgggccttatgaagttggtggagagactgagcatgatgaaagtctcgttcctgttcctgctaacagttacttaggtttttctttggactcagggaaaggtattgtttctaaagatgagatcacttttgtatctggtgctcccagagccaatcacagtggagccgtggttttgctgaagagagacatgaagtctgcacatctcctccctgagcacatattcgatggagaaggtctggcctcttcatttggctatgatgtggcggtggtggacctcaacaaggatgggtggcaagatatagttattggagccccacagtattttgatagagatggagaagttggaggtgcagtgtatgtctacatgaaccagcaaggcagatggaataatgtgaagccaattcgtcttaatggaaccaaagattctatgtttggcattgcagtaaaaaatattggagatattaatcaagatggctacccagatattgcagttggagctccgtatgatgacttgggaaaggtttttatctatcatggatctgcaaatggaataaataccaaaccaacacaggttctcaagggtatatcaccttattttggatattcaattgctggaaacatggaccttgatcgaaattcctaccctgatgttgctgttggttccctctcagattcagtaactattttcagatcccggcctgtgattaatattcagaaaaccatcacagtaactcctaacagaattgacctccgccagaaaacagcgtgtggggcgcctagtgggatatgcctccaggttaaatcctgttttgaatatactgctaaccccgctggttataatccttcaatatcaattgtgggcacacttgaagctgaaaaagaaagaagaaaatctgggctatcctcaagagttcagtttcgaaaccaaggttctgagcccaaatatactcaagaactaactctgaagaggcagaaacagaaagtgtgcatggaggaaaccctgtggctacaggataatatcagagataaactgcgtcccattcccataactgcctcagtggagatccaagagccaagctctcgtaggcgagtgaattcacttccagaagttcttccaattctgaattcagatgaacccaagacagctcatattgatgttcacttcttaaaagagggatgtggagacgacaatgtatgtaacagcaaccttaaactagaatataaattttgcacccgagaaggaaatcaagacaaattttcttatttaccaattcaaaaaggtgtaccagaactagttctaaaagatcagaaggatattgctttagaaataacagtgacaaacagcccttccaacccaaggaatcccacaaaagatggcgatgacgcccatgaggctaaactgattgcaacgtttccagacactttaacctattctgcatatagagaactgagggctttccctgagaaacagttgagttgtgttgccaaccagaatggctcgcaagctgactgtgagctcggaaatccttttaaaagaaattcaaatgtcactttttatttggttttaagtacaactgaagtcacctttgacaccccatatctggatattaatctgaagttagaaacaacaagcaatcaagataatttggctccaattacagctaaagcaaaagtggttattgaactgcttttatcggtctcgggagttgctaaaccttcccaggtgtattttggaggtacagttgttggcgagcaagctatgaaatctgaagatgaagtgggaagtttaatagagtatgaattcagggtaataaacttaggtaaacctcttacaaacctcggcacagcaaccttgaacattcagtggccaaaagaaattagcaatgggaaatggttgctttatttggtgaaagtagaatccaaaggattggaaaaggtaacttgtgagccacaaaaggagataaactccctgaacctaacggagtctcacaactcaagaaagaaacgggaaattactgaaaaacagatagatgataacagaaaattttctttatttgctgaaagaaaataccagactcttaactgtagcgtgaacgtgaactgtgtgaacatcagatgcccgctgcgggggctggacagcaaggcgtctcttattttgcgctcgaggttatggaacagcacatttctagaggaatattccaaactgaactacttggacattctcatgcgagccttcattgatgtgactgctgctgccgaaaatatcaggctgccaaatgcaggcactcaggttcgagtgactgtgtttccctcaaagactgtagctcagtattcgggagtaccttggtggatcatcctagtggctattctcgctgggatcttgatgcttgctttattagtgtttatactatggaagtgtggtttcttcaagagaaataagaaagatcattatgatgccacatatcacaaggctgagatccatgctcagccatctgataaagagaggcttacttctgatgcatag

SEQ ID NO: 4 - DNA, 19 bp, mouse
gagctgtaccgagtgcatc

SEQ ID NO: 5 - RNA, 19 nt, artificial
  RNA inhibitor for B4 integrin chain
gagcugcacggaguguguc

SEQ ID NO: 6 - DNA, 21 bp, mouse
aagagctgtaccgagtgcatc

SEQ ID NO: 7 - DNA, 21 bp, artificial
  integrin b4 amplification primer
ggaaatagcagagcaggatac

SEQ ID NO: 8 - DNA, 20 bp, artificial
  intergin b4 amplification primer
ctcgtgctttacggtatcgc

SEQ ID NO: 9 - DNA, 19 bp, artificial
  integrin b4 amplification primer
ctcggttgcagcaaggaag